SEQUENCE LISTING (3 sequences)

SEQ ID NO: 1 - DNA, 3843 bp, Artificial Sequence
  Nucleotide sequence of SARS-CoV-2 spike region synthetic construct 1
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataataggtcgacgctagcctcgag

SEQ ID NO: 2 - DNA, 3822 bp, Artificial Sequence
  Nucleotide sequence of SARS-CoV-2 spike region synthetic construct 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - DNA, 3813 bp, Artificial Sequence
  Nucleotide sequence of SARS-CoV-2 spike region synthetic construct 3
atgtttgtctttctcgtgctcctccctctcgtctcatcccagtgcgtgaacctgactactcggacccagctccctcccgcttacaccaactccttcacaagaggagtgtattaccccgacaaggtgtttagaagctccgtgctgcactctacacaggatctgtttctgcctttctttagcaacgtgacctggttccacgctatccacgtgtctggcaccaacggaacaaagaggttcgccaacccagtgctgccctttaacgatggcgtgtacttcgcctctaccgagaagagcaacatcatcagaggctggatctttggaaccacactggactccaagacacagtctctgctgatcgtgaacaacgccaccaacgtggtcatcaaggtgtgcgagttccagttttgtaacgatccattcctgggcgtgtactaccacaagaacaacaagagctggatggagtccgagtttagagtgtactctagcgccaacaactgcacatttgagtacgtgtcccagcccttcctgatggacctggagggcaagcagggaaacttcaagaacctgcgggagttcgtgtttaagaacatcgacggctactttaagatctactctaagcacaccccaatcaacctggtgcgcggcctgccacagggcttcagcgccctggagccactggtggatctgcccatcggaatcaacatcaccaggtttcagacactgcacagaagctacctgacaccaggcgactcctctagcggatggaccgctggagctgctgcttactacgtgggctacctgcagccccggaccttcctgctgaagtacaacgagaacggaaccatcacagacgctgtggattgcgccctggaccccctgagcgagacaaagtgtacactgaagtcctttaccgtggagaagggcatctaccagactagtaacttccgggtgcagcctaccgagtctatcgtgcgctttcccaacatcacaaacctgtgcccttttggagaggtgttcaacgctacccgcttcgccagcgtgtacgcttggaaccggaagcgcatctctaactgcgtggccgactacagcgtgctgtacaactccgcttctttcagcacctttaagtgctacggcgtgtcccctacaaagctgaacgacctgtgctttaccaacgtgtacgccgattctttcgtgatcaggggagacgaggtgcgccagatcgctcccggccagacaggaaacatcgctgactacaactacaagctgcctgacgatttcaccggctgcgtgatcgcctggaacagcaacaacctggattccaaagtgggcggaaactacaactacctgtacaggctgtttagaaagagcaacctgaagccattcgagcgggacatctctacagagatctaccaggctggaagcaccccatgcaacggagtgaagggcttcaactgttacttccctctgcagtcctacggattccagccaacatacggcgtgggataccagccctaccgcgtggtggtgctgagctttgagctgctgcacgctcctgctacagtgtgcggaccaaagaagtccaccaacctggtgaagaacaagtgcgttaacttcaactttaacggactgaccggcacaggagtgctgaccgagtccaacaagaagttcctgccttttcagcagttcggccgggacatcgccgataccacagacgctgtgcgcgaccctcagaccctggagatcctggacatcacaccatgctctttcggcggagtgagcgtgatcacaccaggaaccaacacaagcaaccaggtggccgtgctgtaccagggcgtgaactgtaccgaggtgcccgtggctatccacgccgatcagctgacccctacatggagggtgtacagcaccggctccaacgtgttccagacaagagccggctgtctgatcggagctgagcacgtgaacaactcctacgagtgcgacatccctatcggcgccggaatctgtgcttcttaccagacccagacaaactctccaaggagagccaggagcgtggcttcccagtctatcatcgcctacaccatgtccctgggcgtggagaactctgtggcttactctaacaacagcatcgctatccctaccaacttcacaattagcgtgaccacagagatcctgccagtgtccatgaccaagacatctgtggactgcacaatgtacatctgtggagattctaccgagtgcagcaacctgctgctgcagtacggcagcttttgtacccagctgaacagagccctgacaggaatcgctgtggagcaggacaagaacacacaggaggtgttcgcccaggtgaagcagatctacaagaccccccctatcaaggactttggcggattcaacttttcccagatcctgcccgatccttccaagccatctaagaggagctttatcgaggacctgctgttcaacaaggtgaccctggctgatgccggcttcatcaagcagtacggcgattgcctgggagacatcgctgccagggacctgatctgtgcccagaagtttaacggactgaccgtgctgccacccctgctgacagatgagatgatcgctcagtacacaagcgccctgctggctggaaccatcacatccggatggaccttcggcgctggagctgccctgcagatcccctttgccatgcagatggcttaccggttcaacggcatcggagtgacccagaacgtgctgtacgagaaccagaagctgatcgccaaccagtttaactccgctatcggcaagatccaggacagcctgtcctctacagcttccgccctgggaaagctgcaggatgtggtgaaccagaacgctcaggccctgaacaccctggtgaagcagctgagctccaacttcggcgccatctctagcgtgctgaacgacatcctgagcaggctggaccctccagaggctgaggtgcagatcgacaggctgatcacaggaagactgcagtctctgcagacctacgtgacacagcagctgatcagggctgctgagatcagggctagcgccaacctggctgccaccaagatgtccgagtgcgtgctgggccagtctaagagagtggacttttgtggcaagggataccacctgatgtccttcccccagtctgcccctcacggagtggtgtttctgcacgtgacctacgtgccagctcaggagaagaacttcaccacagctcccgccatctgccacgatggcaaggcccactttcctcgggagggcgtgttcgtgtccaacggaacccactggtttgtgacacagcgcaacttctacgagccacagatcatcaccacagacaacacattcgtgtctggcaactgtgacgtggtcatcggaatcgtgaacaacaccgtgtacgatcctctgcagccagagctggacagctttaaggaggagctggataagtacttcaagaaccacacctcccccgacgtggatctgggcgacatcagcggaatcaacgcctccgtggtgaacatccagaaggagatcgacaggctgaacgaggtggctaagaacctgaacgagagcctgatcgatctgcaggagctgggcaagtacgagcagtacatcaagtggccttggtacatctggctgggcttcatcgccggactgatcgctatcgtgatggtgaccatcatgctgtgctgtatgacatcctgctgttcttgcctgaagggctgctgtagctgtggatcctgctgtaaattcgatgaggacgattccgagcctgtgctgaagggcgtgaaactccattacacctga

(19) United States
(12) Patent Application Publication
(10) Pub. No.: US 2024/0123053 A1
(43) Pub. Date: Apr. 18, 2024

(54) CORONAVIRUS VACCINE THROUGH NASAL IMMUNIZATION

(71) Applicant: BHARAT BIOTECH INTERNATIONAL LIMITED, Hyderabad (IN)

(72) Inventors: Krishna Murthy ELLA, Hyderabad (IN); Krishna Mohan VADREVU, Hyderabad (IN); Raches ELLA, Hyderabad (IN); Raju SUNAGAR, Hyderabad (IN)

(73) Assignee: BHARAT BIOTECH INTERNATIONAL LIMITED, Hyderabad (IN)

(21) Appl. No.: 17/923,415

(22) PCT Filed: May 7, 2021
(86) PCT No.: PCT/IN2021/050444

§ 371 Date: Nov. 4, 2022

(30) Foreign Application Priority Data
May 7, 2020 (IN) 202041019441

(51) Int. Cl. A61K 39/215; A61P 37/04; C07K 14/005

(52) U.S. Cl. CPC A61K 39/215; A61P 37/04; C07K 14/005; A61K 2039/5256

(57) ABSTRACT

The invention generally discloses coronavirus vaccine for coronavirus disease. Particularly, the invention discloses coronavirus vaccine through nasal immunization. More particularly, the invention describes and develop a preventive vaccine against infection or disease caused by severe acute respiratory syndrome coronavirus 2 (SARS-CoV-2) through nasal immunization in mammals. Specifically, the invention describes human adenovirus which is engineered to express SARS-CoV-2 spike protein or part/fragment thereof which elicit immune response against the SARS-CoV-2 in mammals, and it is also suitable for immunizing human subjects. Describes the method of production of novel adenovirus vectors, use thereof in vaccine composition, vaccine formulation, preparation, and method of treatment of COVID-19 using above said novel vectors and compositions thereof.

### RELATED PATENT APPLICATION

This application claims the priority to and benefit of Indian Patent Application No. 202041019441 filed on May 7, 2020; the disclosures of which are incorporated herein by reference.

### FIELD OF THE INVENTION

The present invention relates to coronavirus vaccine through nasal immunization for coronavirus disease and/or COVID-19. More particularly, the invention relates to developing a preventive vaccine against infection or disease caused by severe acute respiratory syndrome coronavirus 2 (SARS-CoV-2) through nasal immunization in mammals. The invention describes vaccine candidates produced by adenovirus vector(s) expression which is/are engineered to express SARS-CoV-2 spike protein or part/fragment thereof. More specifically, the invention describes viral vector-based vaccine candidates prepared using human adenovirus type 5 (Ad5) and Chimpanzee adenovirus 36 (ChAd36). Immunogenic or vaccine composition using said vaccine candidates, its production and formulation, its immunogenicity in animals through various routes of administration and method of treatment and/or prophylaxis are described. Also discloses a method to estimate the infectious unit by detection of the transgene (spike) expression by the adenovirus vector.

### BACKGROUND OF THE INVENTION

Coronavirus Disease (COVID-19):

The current outbreak of coronavirus disease was first reported from Wuhan, China, in December 2019. This epidemic had spread to 220 countries and territories around the world with 148,497,686 confirmed cases, including 3,133,860 deaths, as of Apr. 27, 2021. The World Health Organization (WHO) declared it as a Public Health Emergency of worldwide. Clinical symptoms include fever, dry cough, dyspnea, fatigue/tiredness, loss of taste or smell, sore throat, muscle or joint pain, pain or pressure in chest, shortness of breath, chills or dizziness and headache. In 5% of cases the disease can lead to pneumonia, respiratory failure, septic shock, and death.

SARS-CoV-2:

The novel severe acute respiratory syndrome coronavirus (SARS-CoV-2) is an emerging pathogen, which belongs to the genus Betacoronavirus of family Coronaviridae. The genome is linear single-stranded positive sense RNA of approximately 29 Kilobases in size. Coronaviruses are enveloped, spherical in shape and are about 120 nm in diameter. As the genome is positive sense, it is infectious and acts as viral messenger RNA. Coronavirus viruses are found in a wide 20 variety of animals and can cause respiratory and enteric disorders of diverse severity. The genome analysis reveals that the SARS-CoV-2 is closely related to the bat coronavirus than the SARS-CoV or MERS-CoV.

Vaccines Against COVID-19:

Though SARS outbreak occurred in 2002-2003, there are still no vaccines available to prevent SARS-CoV infection. Similarly, vaccine against MERS is also not available. However, a number of SARS- and MERS-CoV vaccine candidates have been tested in animal models, including whole-inactivated, subunit, DNA, mRNA, viral-vectored, and live attenuated vaccines. Most of these vaccine candidates elicit neutralizing antibodies to the coronaviruses spike (S) protein, the major viral antigen. Neutralizing antibodies to the coronavirus spike protein are protective. However, T-cell mediated immune responses also appear to play a role in protection from lethal coronavirus challenges. An ideal vaccine candidate for coronavirus should elicit mucosal, humoral and cell mediated immune response to prevent coronavirus infection.

The current patent disclosure describes the preparation and formulation of vaccine candidates to prevent SARS-CoV-2 infections.

Viral vectored vaccines have been the best vaccines among the novel approaches. Adeno, pox and herpes viruses expressing spike protein of SARS-CoV-2. The best among the vectored vaccines have been the adenovirus vectors, mostly on the background of human adenovirus type 5 (Ad5). Adenoviruses present several advantages: (a) high expression of the transgenes, (b) multiple species tropism (of Ad5), (c) easy construction and manipulation, (d) capacity to clone up to 8 kbp of DNA, (e) their natural tropism to mucosal surfaces (similar to SARS-CoV-2), (f) mild, if any, infection, even in the natural host, (g) compatibility with tests to differentiate vaccinated and infected animals (because non-structural genes can be excluded), (h) ability to induce both humoral and cell-mediated immunity, and (i) compatibility with other vaccines, including classical killed vaccines, in prime boost strategies (Rodriguez and Grubman 2009). Importantly, they can be used to induce early protective immunity mediated by both CD4+ and CD8+ T cells.

The below invention deals with novel adenovirus vectored vaccine constructs for use as vaccine against SARS-CoV-2 in humans.

### OBJECTS OF THE INVENTION

Primary objective of the invention is to provide coronavirus vaccine through nasal immunization for coronavirus disease.

Another objective of the invention to provide a stable immunogenic composition for prophylaxis and eliciting an immune response against SARS-CoV-2 and its variants in mammals including human subjects.

Another objective of the invention is to develop a preventive vaccine against infection or disease caused by severe acute respiratory syndrome coronavirus 2 (SARS-CoV-2) through various routes of administration including nasal immunization in mammals.

Another objective of the invention is to provide adenovirus vectored based coronavirus vaccine formulation in liquid form such as liquid drops or spray or like for vaccination through nasal immunization for COVID-19 which is caused by SARS-CoV-2.

Another objective of the invention is to provide engineered adenovirus to express SARS-CoV-2 spike protein or part/fragment thereof and method/system for production thereof. Use of adenovirus vectors to express spike glycoprotein of SARS-CoV-2 and its variants in mammals.

Another objective of the invention is to provide a vaccine candidate, wherein a human adenovirus type 5 (Ad5) is engineered to express SARS-CoV-2 spike protein or part/fragment which elicits immune response against the SARS-CoV-2 in mammals, and it is also suitable for immunizing human subjects.

Another objective of the invention is to provide a vaccine candidate, wherein a Chimpanzee adenovirus 36 (ChAd36) is engineered to express SARS-CoV-2 spike protein or part/fragment which elicits immune response against the SARS-CoV-2 in mammals, and it is also suitable for immunizing human subjects.

Another objective of the invention is to provide a vaccine that can be used to elicit immune responses against SARS-CoV-2 in humans and can be used against COVID-19.

Another objective of the invention is to provide a method for producing and obtaining the vaccine formulations for recombinant replication-defective adenovirus expressing partial (part or fragment), chimeric, designer, complete or subunit spike glycoprotein of the SARS-CoV-2.

A further objective of the invention is to provide a stable vaccine composition comprising or expressing one or more of the antigens (partial, chimeric, designer, RBD alone or complete or subunit spike glycoprotein of the SARS-CoV-2) in various systems.

It is the objective of the present invention to provide stable immunogenic or vaccine composition using said vaccine candidates, its production and formulation, its immunogenicity in animals through various routes of administration, specifically intranasal administration.

A method of treatment and/or prophylaxis and eliciting an immune response against SARS-CoV-2 and its variants in mammals including human subject, wherein the said method comprises nasal administration of an immunogenic composition or vaccine composition or vaccine formulation of present invention as described using novel adenovirus vectors rAd5-S, rAd5-S1 and ChAd36.

Still, a further objective is to provide a method to estimate the infectious unit by detection of the transgene (spike) expression by the adenovirus vector.

### SUMMARY OF THE INVENTION

The present invention discloses coronavirus vaccine through nasal immunization for coronavirus disease. More particularly, the invention discloses development of a preventive vaccine against infection or disease caused by severe acute respiratory syndrome coronavirus 2 (SARS-CoV-2) through nasal immunization in mammals. The invention describes vaccine candidates produced by adenovirus vector(s) expression which is/are engineered to express SARS-CoV-2 spike protein or part/fragment thereof. Specifically, the invention describes viral vector-based vaccine candidates prepared using human adenovirus type 5 (Ad5) and Chimpanzee adenovirus 36 (ChAd36) which adenoviruses are engineered to express SARS-CoV-2 spike protein or part/fragment thereof, which vaccine candidates elicit immune response against the SARS-CoV-2 in mammals, and these vaccine candidates are also suitable for immunizing human subjects. Immunogenic or vaccine composition using said vaccine candidates, its production and formulation, its immunogenicity in animals through various routes of administration are described. Also discloses a method to estimate the infectious unit by detection of the transgene (spike) expression by the adenovirus vector.

The present invention provides a novel adenovirus vectored vaccine construct for use as vaccine against SARS-CoV-2 in humans. The present invention provides a stable immunogenic vaccine composition for prophylaxis and eliciting an immune response against SARS-CoV-2 and its variants through nasal immunization. The said vaccine is an adenovirus vectored vaccine for Covid-19. In one embodiment, said adenovirus is the adenovirus type-5. The adenovirus type 5 is engineered to express SARS-CoV-2 spike protein. The said adenovirus vectors express the spike glycoprotein of SARS-CoV-2 and its variants.

In the said vaccine composition, the expressed spike glycoprotein is one of complete, partial or chimeric, wherein the spike glycoprotein of the SARS-CoV-2 are expressed under transcriptional control. The said transcriptional control is any transcriptional control element introduced into the viral vector expressing spike glycoprotein of SARS-CoV-2 and the transcriptional control elements include lac operator, tet operator and similar operators. The said transcriptional control reduce the expression of spike glycoprotein of the SARS-CoV-2 during generation and propagation of virus vectors expressing spike glycoprotein of the SARS-CoV-2 in mammalian cells specifically developed for controlled expression of SARS-CoV-2 virus proteins under the control elements.

The said cells are mammalian cells developed for expression of a transcription control protein suitable to control transcription of spike glycoprotein of the SARS-CoV-2 under transcriptional elements. The said spike glycoprotein of the SARS-CoV-2 includes complete spike glycoprotein or truncated or designer spike or receptor binding alone. The adenovirus carrying SARS-CoV-2 genes is generated by recombination between an adenovirus genomic plasmid and the shuttle plasmid containing the SARS-CoV-2 genes, and recombination between the plasmids is mediated by a recombinase, which is expressed from the genomic plasmid.

The said composition does not induce antibodies to non-structural proteins of SARS-CoV-2, so that tests designed to differentiate vaccinated and infected humans based on the detection of antibodies to certain non-structural proteins can be employed in combination with immunization with the above said composition.

In the above said and further below described composition the adenovirus vector is at the concentration between 10\hat{ }9 to 10\hat{ }12 (i.e., 10⁹-10¹²) virus particles.

The said adenovirus vector (rAd5-S, rAd5-S1, ChAd-S) is prepared by the process comprising lysis of the infected cells, combination of the TFF, Hollow fiber or size-exclusion chromatography.

In yet another aspect, the present invention provides a method of production and obtaining the vaccine formulations for recombinant replication-defective adenovirus expressing partial, chimeric, designer or complete or subunit spike glycoprotein of the SARS-CoV-2 which comprises:
a) Using 293 or 293 IQ (or any other cell line expressing E1 region of replication-defective adenovirus) for propagation of recombinant replication-defective adenovirus expressing partial, chimeric or complete spike glycoprotein of the SARS-CoV-2 or S1 subunit. The obtained recombinant adenovirus is an immunogenic composition to prevent disease and/or infection with SARS-CoV-2.

In yet another aspect, the present invention provides a method to estimate the infectious unit by detection of the transgene (spike) expression by the adenovirus vector by chromogenic substrate, wherein chromogenic substrate is 3-Amino-9-Ethylcarbazole (AEC) or 3,3′-Diaminobenzidine (DAB).

In yet another aspect, the present invention provides a stable vaccine composition comprising or expressing one or more of the antigen (partial, chimeric, designer, RBD alone or complete spike glycoprotein of the SARS-CoV-2) in various systems, the said antigens being formulated in pharmaceutically acceptable buffer, wherein the vaccine composition elicits protective immune response to SARS-CoV-2 in mammals when administered through intranasal (I/N), oral, intramuscular (I/M), subcutaneous, intradermal and transcutaneous routes.

In the said stable immunogenic composition, the active ingredient adenovirus vector is in the range between 10\hat{ }9 to 10\hat{ }12 virus particles and are buffered with Tris-HCl at pH from 7.0 to 7.6 of concentration 10 to 20 mM, Glycerol as cryo-protectant of concentration between 1.5 to 4.5%, Sodium chloride for osmolarity of concentration between 20 to 30 mM, Magnesium chloride as stabilizer of concentration between 1 to 4 mM and Polysorbate-80 as stabilizer of concentration between 0.01 to 0.2%. Preferably the said composition may be administered intranasally (IN). Other routes may be such as orally, intramuscularly (UM), subcutaneously, intradermally, intravenously, vaginally or any other route, either by a single route or any of the combinations.

A coronavirus vaccine composition for prophylaxis and eliciting an immune response against SARS-CoV-2 and its variants in mammals, the said vaccine composition comprises an immunogenic composition comprising one or more viral vector(s) engineered to express SARS-CoV-2 spike protein.

The said vaccine composition, wherein the vaccine is adenovirus vectored vaccine for Covid-19, wherein the viral vector used is adenovirus. Said adenovirus viral vector used in the expression system may be Human adenovirus, Chimpanzee adenovirus or other suitable adenovirus species. The said adenovirus is human adenovirus type-5 (Ad5) or Chimpanzee adenovirus 36 (ChAd36) engineered to express SARS-CoV-2 spike glycoprotein.

Use of adenovirus vector to express spike glycoprotein of SARS-CoV-2 and its variants in mammals.

The said vaccine composition, wherein the said spike glycoprotein of SARS-CoV-2 which is expressed in adenovirus may be full length spike or parts thereof including RBD alone or designer or truncated or partial or chimeric spike or S1 subunit or S2 subunit.

The said spike glycoprotein of SARS-CoV-2 which is expressed in adenovirus Ad5 or ChAd36 is
full-length (S) SARS-CoV-2 spike protein (SARS-CoV2-S) having nucleotide sequence of SEQ. ID No. 1, or
subunit (S1) of SARS-CoV-2 spike protein (SARS-CoV2-S1) having nucleotide sequence of SEQ. ID No. 2, or
full-length (S) SARS-CoV-2 spike protein (SARS-CoV2-S) having nucleotide sequence of SEQ. ID No. 3.

The said vaccine composition, wherein the expression of spike glycoprotein of SARS-CoV-2 in adenovirus Ad5 or ChAd36 produces below adenovirus vector constructs 1-3 (vaccine candidates 1-3):

Construct-1:
Ad5 expresses full-length(S) SARS-CoV-2 spike protein (SARS-CoV2-S)

Construct-2:
Ad5 expresses subunit (S1) of SARS-CoV-2 spike protein (SARS-CoV2-S1)

Construct-3:
ChAd36 expresses full-length(S) SARS-CoV-2 spike protein (SARS-CoV2-S).

The said vaccine composition, wherein said adenoviruses-based vector construct-1, construct-2, and construct-3, produce adenovirus vectors rAd5-S, rAd5-S1, and ChAd36 respectively, which are used as vaccine candidates (antigens) for preparation of vaccine for mammals for Covid-19.

In the said vaccine composition, the said spike glycoprotein of the SARS-CoV-2 are expressed under transcriptional control. Said transcriptional control is any transcriptional control element introduced into the viral vector expressing spike glycoprotein of SARS-CoV-2. The said transcriptional control elements include but are not limited to lac operator, tet operator and other similar operators. The said transcriptional control reduce the expression of spike glycoprotein of the SARS-CoV-2 during generation and propagation of virus vectors expressing spike glycoprotein of the SARS-CoV-2 in mammalian cells specifically developed for controlled expression of SARS-CoV-2 virus proteins under the control elements as stated above.

The above said cells are mammalian cells developed for expression of a transcription control protein suitable to control transcription of spike glycoprotein of the SARS-CoV-2 under transcriptional elements.

The said immunogenic composition of vaccine comprises one or more antigen(s) selected from adenovirus vectors rAd5-S, rAd5-S1, and ChAd36 in a concentration in the range between 10\hat{ }9 to 10\hat{ }12 virus particles. The composition may comprise and/or formulated with or without one or more pharmaceutically acceptable excipient(s) which may be selected from buffer, cryo-protectant, salt, isotonic agent, stabilizer, diluent or carrier, propellant (for spray formulation) etc. The composition is typically formulated as a liquid, solution, microemulsion, emulsion, liposome, spray or other formulation type suitable for administration to a human subject, preferably the composition is formulated in a suitable dosage form for intranasal (I/N) or intramuscular (UM) administration. In one embodiment the composition is formulated as a liquid formulation or a spray formulation, suitable for intranasal administration.

Use of novel adenovirus vector rAd5-S or rAd5-S1 or ChAd36 as antigen in a concentration range between 10\hat{ }9 to 10\hat{ }12 virus particles.

Vaccine composition, wherein the immunogenic composition comprises following ingredients:
Antigen: active ingredient adenovirus vector selected from rAd5-S or rAd5-S1 or ChAd36 or any combination thereof as antigen(s) in a concentration range between 10\hat{ }9 to 10\hat{ }12 virus particles;
Buffer: buffered with Tris-HCl or phosphate buffer or combination thereof at pH that ranges from 7.0 to 7.6 in a concentration range between 10 to 20 mM,
Cryo-protectant: Glycerol in a concentration range between 1.5 to 4.5%,
Salt: Sodium chloride for osmolarity in a concentration range between 20 to 30 mM,
Stabilizer: Magnesium chloride in a concentration range between 1 to 4 mM, and
Stabilizer (optional): Polysorbate-80 in a concentration range between 0.01 to 0.2%.

Vaccine composition, wherein the immunogenic composition comprises following ingredients:
Antigen: active ingredient adenovirus vector selected from rAd5-S or rAd5-S1 or ChAd36 or any combination thereof as antigen(s) in a concentration range between 10\hat{ }9 to 10\hat{ }12 virus particles;
Tris-HCl 20 mM at pH that ranges from 7.0 to 7.6,
Glycerol 2.5%,
Sodium chloride 25 mM,
Magnesium chloride 2 mM, and
Polysorbate-80 at 0.1%.

A method of production of adenovirus vectors rAd5-S, rAd5-S1, ChAd-S, wherein the method involves lysis of the infected cells, combination of the TFF, Hollow fiber or size-exclusion chromatography.

A Stable immunogenic composition, wherein the one or more active ingredient adenovirus vector(s) between 10\hat{ }9 to 10′42 virus particles as antigen(s) are buffered with Tris-HCl at pH that ranges from 7.0 to 7.6 in a concentration range between 10 to 20 mM, Glycerol as cryo-protectant in a concentration range between 1.5 to 4.5%, Sodium chloride for osmolarity in a concentration range between 20 to 30 mM, Magnesium chloride as stabilizer in a concentration range between 1 to 4 mM, and with or without Polysorbate-80 as stabilizer in a concentration range between 0.01 to 0.2%. The stable immunogenic composition is administered intranasally (I/N).

Novel adenovirus vector viz. rAd5-S or rAd5-S1 or ChAd36 to be used as antigen in the preparation of adenovirus viral vector-based vaccine for Covid-19 and prophylaxis and eliciting an immune response against SARS-CoV-2 and its variants in mammals, wherein the said adenovirus is adenovirus type-5 (Ad5) or Chimpanzee adenovirus 36 (ChAd36) which is engineered to express SARS-CoV-2 spike glycoprotein and its variants in mammals, and wherein the adenovirus vector comprises a vector construct as follows:

Vector Construct-1:
Ad5 expresses full-length (S) SARS-CoV-2 spike protein (SARS-CoV2-S),

Vector Construct-2:
Ad5 expresses subunit (51) of SARS-CoV-2 spike protein (SARS-CoV2-S1),

Vector Construct-3:
ChAd36 expresses full-length (S) SARS-CoV-2 spike protein (SARS-CoV2-S).

The above said adenovirus vector(s), wherein the said spike glycoprotein of SARS-CoV-2 or parts/fragments thereof which is expressed in adenovirus is full length spike glycoprotein (S) or S1 subunit glycoprotein which comprises:
full-length (S) SARS-CoV-2 spike protein (SARS-CoV2-S) having nucleotide sequence of SEQ. ID No. 1,
or
subunit (51) of SARS-CoV-2 spike protein (SARS-CoV2-S1) having nucleotide sequence of SEQ. ID No. 2,
or
full-length (S) SARS-CoV-2 spike protein (SARS-CoV2-S) having nucleotide sequence of SEQ. ID No. 3.

The above said viral vector is adenovirus which may be selected from other species, including but not limited to bovine, ovine, caprine, porcine, rhesus, chimpanzee or avian adenoviruses, or any other viral vector, in combination with or without the controlled system of expression.

A method of treatment and/or prophylaxis for COVID-19 and/or eliciting an immune response against SARS-CoV-2 and its variants in mammals including human subjects, wherein the said method comprises nasal administration of a vaccine formulation comprising: a vaccine composition, and/or a stable immunogenic composition as stated above.

Optionally, in the above said method of treatment and/or prophylaxis, the above said composition/formulation may be administered in combination with other suitable immunogenic composition which may be selected from immunogenic compositions for various types of live or replication-defective Influenza vaccine compositions.

The vaccine composition, immunogenic composition and method of treatment as described above, wherein the composition or vaccine is formulated in a suitable dosage form, stored in a suitable container suitable for nasal or intranasal application to human subjects in a suitable dose sufficient to elicit an immune response against SARS-CoV-2 and its variants in mammals. The suitable dosage form may comprise of liquid of drop(s) or spray form or other similar forms suitable for intranasal (I/N) administration.

In yet another aspect, the SARS-CoV-2 antigen compositions (partial, chimeric or complete or subunit spike glycoprotein) provided in the present invention is effective against any betacoronavirus strains that share anywhere between 50% to 100% identity at any other region of the genome particularly in the amino acid levels in the receptor binding domain of the spike glycoprotein.

A method to estimate the infectious unit by detection of the transgene (spike) expression by the adenovirus vector by chromogenic substrate but not limited to 3-Amino-9-Ethylcarbazole (AEC) or 3,3′-Diaminobenzidine (DAB) that correlates to the expression of the transgene (spike protein) which can be extrapolated to the detection of the quantity of spike expressing units in the sample.

Adenovirus vectors, their production, composition, formulation and its production, immune response and method of estimation as described in detail below description, examples, shown and represented in figures and as claimed in appended claims.

### BRIEF DESCRIPTION OF DRAWINGS

FIG. 1: Diagrammatic representation of SARS-CoV-2 genome (Example-1).

FIG. 2: Schematic representation of the different spike gene constructs incorporated in the adenovirus (Example-3).

FIG. 3: Schematic representation of the control elements to regulate protein expression via the recombinant adenovirus (Example-4).

FIG. 4: Schematic representation of the Ad-Max HiIQ system of generation of recombinant adenoviruses (Example-5).

FIG. 5: Expression and purification of SARS-CoV-2 spike protein in E. coli (Example-6).

FIG. 6: Expression of spike protein (S) by rAd5-Spike (Example-7).

FIG. 7: Expression of subunit (S1) by rAd5-S1 (Example-8).

FIG. 8: Expression of spike protein (S) by ChAd-S (Example-9).

FIG. 9: Growth Kinetics of rAd5-S (Example-10).

FIG. 10: Growth Kinetics of ChAd-S (Example-11).

FIG. 11: Process workflow for manufacture of adenovirus vectored COVID-19 vaccine (Example-12).

FIG. 12: Lysis methods and adenovirus recovery (Example-13).

FIG. 13: Anion-exchange chromatography of rAd5-Spike (Example-14).

FIG. 14: Anion-exchange chromatography of ChAd-S (Example-15).

FIG. 15: Estimation of infectious units by transgene expression (Example-17).

FIG. 15A: Uninfected 293 cells

FIG. 15B: ChAd-S infected 293 cells—AEC staining

FIG. 15C: ChAd-S infected 293 cells—DAB staining

Antibody Response of Mice Immunized with rAd5-Spike Vector:

FIG. 16: Serum IgG antibody response of mice immunized with rAd5-Spike (Example-18).

FIG. 17: Serum IgA antibody response of mice immunized with rAd5-Spike (Example-19).

FIG. 18: Bronchio-alveolar IgA antibody response of mice immunized with rAd5-Spike (Example-20).

Comparison of Serum IgG and IgA Titers in I/N and I/M Route of Administration:

FIG. 19: Serum IgG titers post-immunization with ChAd-S (Example-21).

FIG. 20: Serum IgA titers post-immunization with ChAd-S (Example-22).

Antibody Response of Mice Immunized with ChAd-S Vector:

FIG. 21: Serum IgG antibody response of mice immunized with ChAd-S vector (Example-23).

FIG. 22: Neutralizing antibody response of mice immunized with ChAd-S vector (Example-24).

### DETAILED DESCRIPTION OF THE INVENTION

The present invention discloses and describes “coronavirus vaccine through nasal immunization” for coronavirus disease including COVID-19. More particularly, the invention discloses and describes development of a preventive vaccine against infection or disease caused by severe acute respiratory syndrome coronavirus 2 (SARS-CoV-2) through nasal immunization in mammals. The vaccine of the present invention described is a vector-based vaccine produced by recombinant adenovirus.

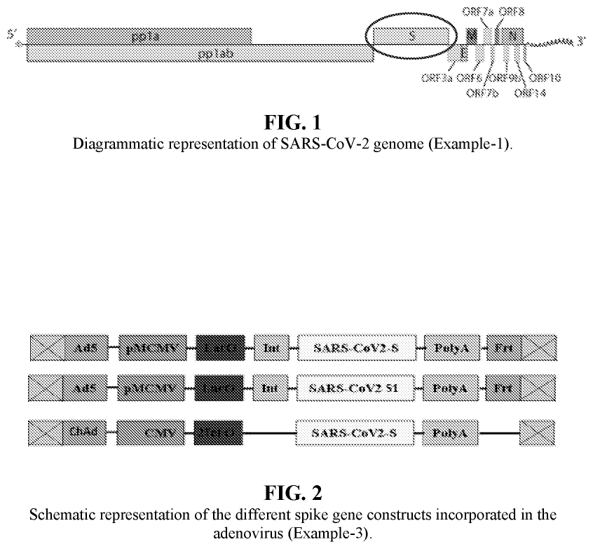

The present invention describes one or more vaccine candidates produced by adenovirus vector(s) expression wherein one or more adenovirus is/are engineered to express SARS-CoV-2 spike protein or part/fragment thereof. Thus, the said vaccine candidates are recombinant adenovirus vector(s) comprising SARS-CoV-2 spike protein or part/fragment thereof. A diagrammatic representation of SARS-CoV-2 genome is shown in FIG. 1. The SARS-CoV-2 genome is a linear, positive sense single stranded RNA of about ˜29000 nucleotides. Like other coronaviruses, SARS-CoV-2 has four structural proteins, known as the S (spike), E (envelope), M (membrane), and N (nucleocapsid) proteins. The spike protein is surface-exposed and mediates entry into host cells, spike is the main target of neutralizing antibodies upon infection and the focus of vaccine design.

Thus, in one aspect the invention describes a coronavirus vaccine composition for prophylaxis and eliciting an immune response against SARS-CoV-2 and its variants in mammals, the said vaccine composition comprises an immunogenic composition comprising one or more viral vector(s) engineered to express SARS-CoV-2 spike protein. In one embodiment the present invention describes viral vector-based vaccine candidates prepared using adenoviruses which are engineered to express SARS-CoV-2 spike protein or part/fragment thereof, which vaccine candidates elicit immune response against the SARS-CoV-2 in mammals, and these vaccine candidates are also suitable for immunizing human subjects.

The present invention also describes generation of an adenovirus vectored vaccine for COVID-19. Immunogenic or vaccine composition using said vaccine candidates, its production and formulation, its immunogenicity in animals through various routes of administration are described.

In one aspect of the present invention the immunogenicity of above said vaccine candidates are evaluated in animals through various routes of administration such as intranasal (I/N) and/or intramuscular (UM) routes.

Adenovirus:

The present invention uses adenovirus vector expression system. The adenovirus used in the expression system may be “human adenovirus”, “Chimpanzee adenovirus” or any other suitable adenovirus species. The viral vector can be an adenovirus from other adenovirus species, including but not limited to bovine, ovine, caprine, porcine, rhesus, chimpanzee or avian adenoviruses, or any other viral vector, in combination with the controlled system of expression as outlined above. Preferably, the viral vector is “human adenovirus” and/or “Chimpanzee adenovirus”.

Human Adenovirus:

The term “human adenovirus” as used herein may include all human adenoviruses of the Adenoviridae family. In one embodiment, the adenovirus can be of serogroup A, B, C, D, E, or F. The human adenovirus can be selected from a serotype of other known serotypes of Adenovirus. In one preferred embodiment of present invention, the said “human adenovirus” specifically selected and used is Adenovirus serotype 5 (Ad5). In one preferred embodiment of the present invention Ad5 is used to produce the vaccine candidate which express stabilized SARS-CoV-2 spike protein or part/fragment thereof.

Chimpanzee Adenovirus:

The term “Chimpanzee adenovirus” as used herein comprises a “Chimpanzee adenovirus 36 (ChAd36)”. In one preferred embodiment of the present invention ChAd36 is used to produce the vaccine candidate which express stabilized SARS-CoV-2 spike protein or part/fragment thereof.

Specifically, the invention describes a “human adenovirus type 5 (Ad5)” and “a Chimpanzee adenovirus 36 (ChAd36)” which are engineered to express “SARS-CoV-2 spike protein” which elicit immune response against the SARS-CoV-2 in mammals, and it is also suitable for immunizing human subjects. The said “SARS-CoV-2 spike protein” for expression may be “full length spike” or parts thereof, especially, RBD alone or designer or truncated or partial or chimeric spike or S1 subunit or S2 subunit.

Specifically, the invention describes viral vector-based vaccine candidates prepared using human adenovirus type 5 (Ad5) and/or Chimpanzee adenovirus 36 (ChAd36) which adenoviruses are engineered to express SARS-CoV-2 spike protein or part/fragment thereof, which vaccine candidates elicit immune response against the SARS-CoV-2 in mammals, and these vaccine candidates are also suitable for immunizing human subjects. Immunogenic or vaccine composition using said vaccine candidates, its production and formulation, its immunogenicity in animals through various routes of administration are described.

In one aspect the invention disclosed novel adenovirus vector viz. rAd5-S or rAd5-S1 or ChAd36 to be used as antigen in the preparation of adenovirus viral vector-based vaccine for Covid-19 treatment and/or prophylaxis and eliciting an immune response against SARS-CoV-2 and its variants in mammals, wherein the said adenovirus is adenovirus type-5 (Ad5) or Chimpanzee adenovirus 36 (ChAd36) which is engineered to express SARS-CoV-2 spike glycoprotein and its variants in mammals, and wherein the adenovirus vector comprises a vector construct as follows:

In one general embodiment, the invention provides expression of below different spike protein constructs through human adenovirus type 5 (Ad5) and/or Chimpanzee adenovirus 36 (ChAd36)” as a vaccine candidate, viz.
human adenovirus type 5 (Ad5) expresses full-length (S) SARS-CoV-2 spike protein,
human adenovirus type 5 (Ad5) expresses receptor binding domain (RBD) of SARS-CoV-2 spike protein,
human adenovirus type 5 (Ad5) expresses designer (dS) SARS-CoV-2 spike protein,
human adenovirus type 5 (Ad5) expresses subunit (S1) of SARS-CoV-2 spike protein,
chimpanzee adenovirus 36 (ChAd36) expresses full-length (S) SARS-CoV-2 spike protein.

In the above constructs,

“S” stands for full length SARS-CoV-2 spike protein.

“S1” stands for S1 subunit of SARS-CoV-2 spike protein.

“RBD” stands for receptor binding domain (RBD) of SARS-CoV-2 spike protein.

“dS” stands for designer spike protein of SARS-CoV-2 spike protein.

In one preferred embodiment, the invention describes expression of below three different spike protein constructs 1-3 (respectively presented in SEQ. ID No. 1-3) through human adenovirus type 5 (Ad5) or Chimpanzee adenovirus 36 (ChAd36)” as a vaccine candidate, viz.

Vector Construct-1:
Ad5 expresses full-length (S) SARS-CoV-2 spike protein (SARS-CoV2-S),

Vector Construct-2:
Ad5 expresses subunit (51) of SARS-CoV-2 spike protein (SARS-CoV2-S1),

Vector Construct-3:
ChAd36 expresses full-length (S) SARS-CoV-2 spike protein (SARS-CoV2-S).

The said spike glycoprotein of SARS-CoV-2 or parts/fragments thereof which is expressed in adenovirus is full length spike glycoprotein (S) or 51 subunit glycoprotein which comprises:
full-length (S) SARS-CoV-2 spike protein (SARS-CoV2-S) having nucleotide sequence of SEQ. ID No. 1,
   or
subunit (51) of SARS-CoV-2 spike protein (SARS-CoV2-S1) having nucleotide sequence of SEQ. ID No. 2,
   or
full-length (S) SARS-CoV-2 spike protein (SARS-CoV2-S) having nucleotide sequence of SEQ. ID No. 3.

Schematic representation of above three spike gene constructs 1-3 incorporated in the adenovirus are shown and represented in FIG. 2 and further described in Example-3.

The vaccine can be used to elicit immune responses against SARS-CoV-2 in humans. The vaccine is prepared by propagating the recombinant adenovirus (recombinant adenovirus vectors rAd5-S or rAd5-S1 or ChAd36) in 293IQ human embryonic kidney cells. The vaccine virus can be clarified and concentrated or purified, and if required, mixed with one or more suitable buffer composition and/or diluent and/or can be formulated with or without use of one or more pharmaceutically acceptable excipient(s). The immunogenic composition/vaccine composition and vaccine formulation thereof is further described in paragraphs below and in Example-16 and further biological studies are performed in animal models taking the formulated compositions prepared in the present invention.

The immunogenic composition or vaccine composition or vaccine formulation of the present invention (simply “vaccine”) is administered via mucosal routes, preferably via nasal route (intranasal) and for this a suitable dosage form may be selected for the formulation such as liquid or spray formulation. Further, the vaccine can also be prepared in suitable dosage form other than mentioned above to be administered via other mucosal routes (oral, mucosal, vaginal or other routes) or parenterally (intramuscular, subcutaneous, intravenous, intraperitoneal or other routes) to humans and for this suitable dosage form may be selected based on the choice of route selected. Unlike the other adenovirus vectored vaccines, this invention does not use a constitutive gene expression system, but employs a controlled gene expression system. The above said vaccine composition, its production and examples are further described in paragraphs below.

In another aspect the invention discloses novel adenovirus vectors to be used as antigen for vaccine production to be used against COVID-19.

In another aspect the invention discloses a method of production of adenovirus vectors rAd5-S, rAd5-S1, ChAd-S, wherein the method involves lysis of the infected cells, combination of the TFF, Hollow fiber or size-exclusion chromatography.

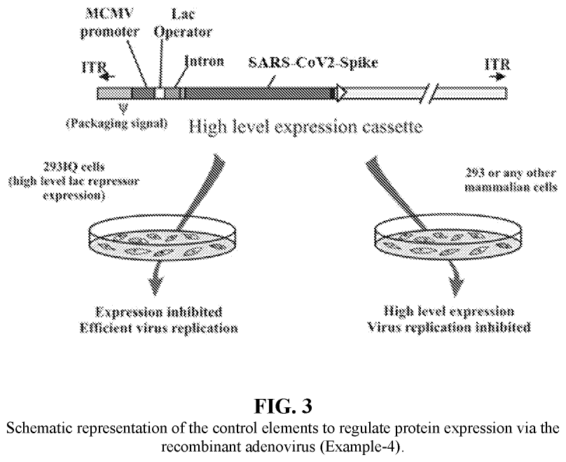

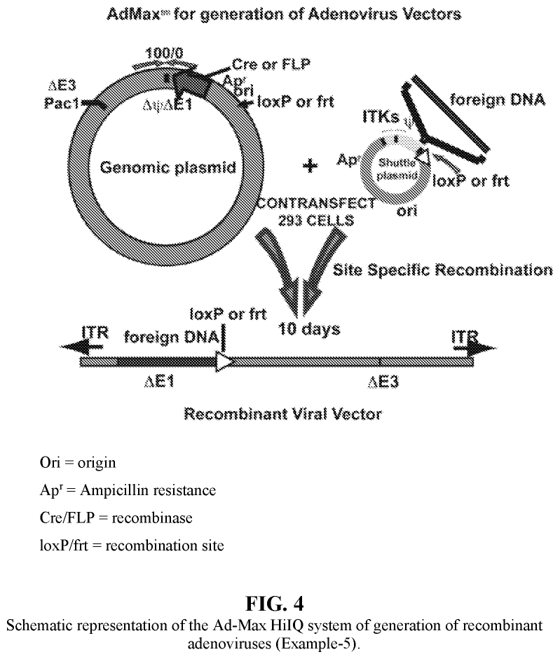

Adenovirus and Production Thereof
1. The recombinant adenovirus is produced using a specialized system, where
   a. The SARS-CoV-2 spike glycoprotein is expressed under lac operator or tet operator controlled promoters. The 293IQ cells express lac repressor and the rAd5-S and/or rAd5-S1 have a lac operator controlled MCMV promoter. In the case of ChAd-S, it has tet operator (2Tet-O) and controlled CMV promoter.
   b. The sequence of SARS-CoV-2 encompasses the region containing spike shown in FIG. 1 and described in Example 1.
      The sequence of the gene encoding the spike protein or parts/fragments thereof (Nucleotide sequence of SARS-CoV-2 spike region synthetic construct 1, construct 2, and construct 3) are provided in SEQ. ID No. 1, SEQ. ID No. 2, and SEQ. ID No. 3 respectively, and described in Example 2. These genes (synthetic constructs 1-3) are incorporated into adenovirus vector (Ad5 or ChAd) as shown in FIG. 2 and explained in Example 3.
   c. The SARS-CoV-2 spike constructs 1-3 are under the control of murine cytomegalovirus immediate early promoter (pMCMV) or cytomegalovirus (CMV), which is immediately downstream of the lac operator (in case of Ad5) or tet operator (in case of ChAd) and an intron (in case of Ad5) is included between the operator and the promoter, as shown in FIG. 2 and Example 3.
   d. A derivative of 293 cell line (293IQ), which stably expresses the bacterial lac repressor protein, is used for the production of the recombinant adenoviruses expressing SARS-CoV-2 spike protein, so that (i) the lac repressor expressed in these cells suppresses the expression of SARS-CoV-2 genes whose promoter is under the lac operator, and (ii) the SARS-CoV-2 proteins can be expressed in any other cell lacking the ability to produce the lac repressor protein, as shown in FIG. 3 and Example 4.
   e. The recombinant adenovirus (rAd5-S or rAd5-S1) carrying SARS-CoV-2 genes (selected from Constructs 1-3, respectively in SEQ. ID No. 1-3) is generated by recombination between an adenovirus genomic plasmid and the shuttle plasmid containing the SARS-CoV-2 genes (selected from Constructs 1-3, respectively in SEQ. ID No. 1-3), and recombination between the plasmids is mediated by a recombinase (Flipase), which is expressed from the genomic plasmid, as shown in FIG. 4 and Example 5.
2. The nucleic acid sequence (SEQ. ID No. 1-3) encoding SARS-CoV-2 proteins can be
   a. Derived from any of the many strains of SARS-CoV-2. The sequence can be derived from “Wuhan strain, B.1.617, B.1.351, B.1.1.7 or any variant of SARS CoV-2”. In one embodiment SEQ. ID No. 1-2 are of Wuhan, SEQ. ID No. 3 is B.1.351—South African Variant.
   b. The full-length spike or parts thereof, especially, RBD alone or designer or truncated or partial or chimeric spike or subunit such as 51 subunit protein are used. More specifically, in one embodiment full-length Spike (S) i.e. SARS-CoV2-S and subunit (51) i.e. SARS-CoV2-S1 spike proteins are used (as shown in FIG. 2).
   c. The above said genes viz. “SARS-CoV2-S” and “SARS-CoV2-S1” of SARS-CoV-2 are obtained entirely synthetically to correspond to the amino acid sequence of the relevant protein or region of SARS-CoV-2.
3. The recombinant adenovirus (rAd5-S or rAd5-S1) or adenovirus ChAd-S is produced in large-scale in cells grown in adherent or suspension systems, or any sequential combination.
   Method: The harvest derived from suspension or adherent culture were lysed, DNA fragmented and clarified which was followed by concentration of up to 10 times. The concentrated samples were further subjected to size exclusion chromatography which is followed by approximately 3 to 5 times concentration to produce the drug substance (recombinant adenovirus vector—which is vaccine candidate).

In another aspect the invention discloses a method of production and obtaining the vaccine formulation. The said method of production and obtaining the vaccine formulations for recombinant replication-defective adenovirus expressing partial, chimeric, designer or complete spike glycoprotein or subunit (S1) of the SARS-CoV-2 which comprises:
a. Using 293 or 293 IQ cells (or any other cell line complementing E1 region of adenovirus) for propagation of recombinant replication-defective adenovirus expressing partial, chimeric or complete spike glycoprotein (S) or subunit (S1) of the SARS-CoV-2.

The said spike glycoprotein of the SARS-CoV-2 are expressed under transcriptional control, wherein said transcriptional control is any transcriptional control element introduced into the viral vector expressing spike glycoprotein of SARS-CoV-2. Said transcriptional control elements include but are not limited to lac operator, tet operator and other similar operators. The said transcriptional control reduce the expression of spike glycoprotein of the SARS-CoV-2 during generation and propagation of virus vectors expressing spike glycoprotein of the SARS-CoV-2 in mammalian cells specifically developed for controlled expression of SARS-CoV-2 virus proteins under the control elements. The said cells are mammalian cells developed for expression of a transcription control protein suitable to control transcription of spike glycoprotein of the SARS-CoV-2 under transcriptional elements.

Vaccine Production Method:

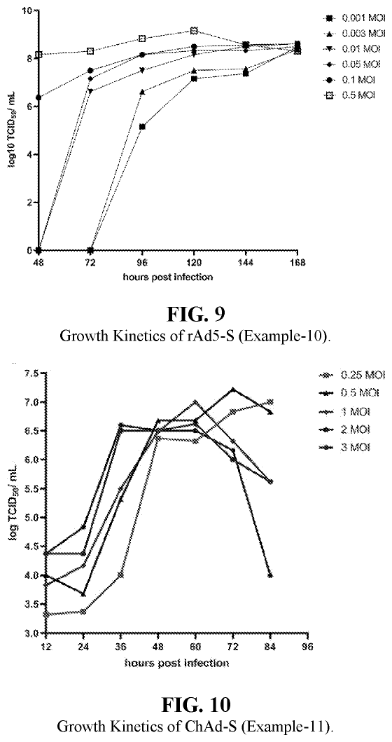

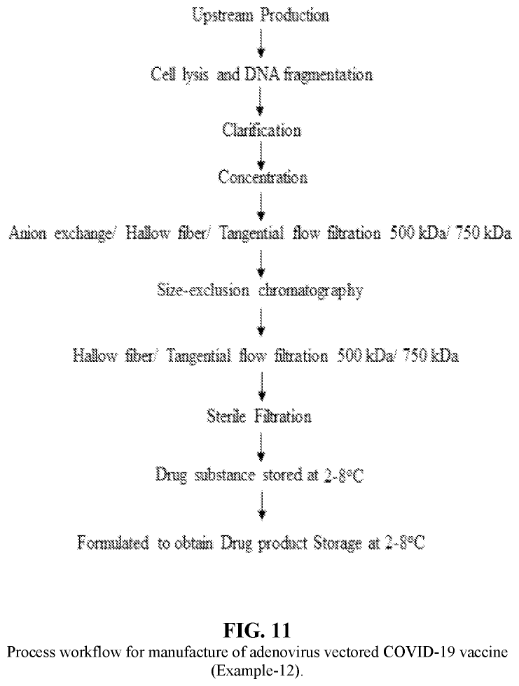

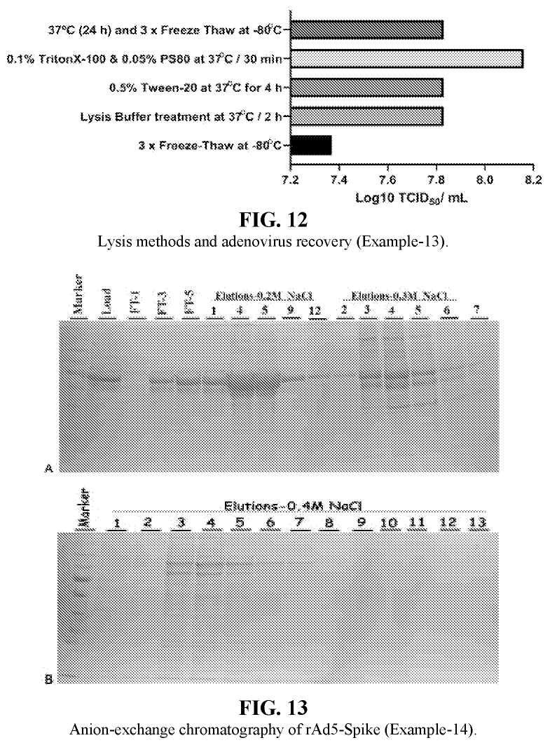

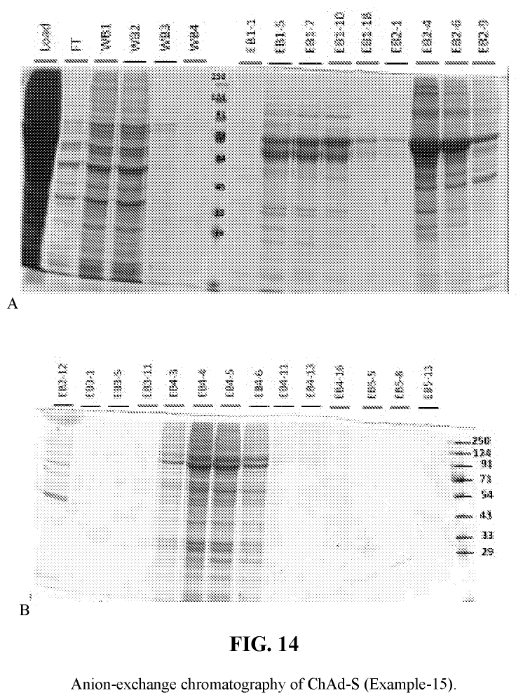

The E1 complementing cells (293 or 293 IQ cells) can be infected as per the multiplicity of infection described in the FIG. 9 and FIG. 10. The cells can be harvested between 60 to 72 hours post infection and lysed with methods as mentioned in FIG. 12. The lysates were then clarified and sterile filtered with depth filters. These samples were either purified with anion exchange chromatography (FIGS. 13 and 14) or concentrated between 10-15 times. The concentrates were further subjected to size exclusion chromatography which is followed by approximately 3 to 5 times concentration to produce the drug substance. The drug substance is diluted with formulation buffer as mentioned in example 16. An outline and workflow for manufacture of adenovirus vectored COVID-19 vaccine of the present invention is shown in FIG. 11 and further described in Examples-12, 16.

In one preferred embodiment, in the above described method of production of recombinant adenovirus vectors, the following viral vectored vaccine candidates are prepared:

| | | | | | Vaccine |
|---|---|---|---|---|---|
| | | SARS-CoV-2 | SEQ. | | Candidates |
| Construct | Adenovirus | spike protein | ID. No. | Expression | Developed |
| Construct-1: | Ad5 | SARS-CoV2-S | 1 | expresses | rAd5-S |
| | | | | full-length (S) | |
| Construct-2: | Ad5 | SARS-CoV2-S1 | 2 | expresses | rAd5-S1 |
| | | | | subunit (S1) | |
| Construct-3: | ChAd36 | SARS-CoV2-S | 3 | expresses | ChAd-S |
| | | | | full-length (S) | |

Vaccine
4. The recombinant adenovirus produced through the above methods is an immunogenic composition or vaccine composition and the composition can be used to prevent disease and/or infection with SARS-CoV-2. In one embodiment the said immunogenic composition comprises one or more antigen(s) selected from adenovirus vectors rAd5-S, rAd5-S1, and ChAd36 in a concentration in the range between 10\hat{ }9 to 10\hat{ }12 virus particles. The thus produced vaccine candidate is further provided as an immunogenic composition or vaccine composition comprising an immunogenically effective concentration of vaccine candidate sufficient to elicit desires result with or without one or more pharmaceutically acceptable excipients(s). The composition may comprise and/or formulated with or without one or more pharmaceutically acceptable excipient(s) which may be selected from buffer, cryo-protectant, salt, isotonic agent, stabilizer, diluent or carrier, propellant (for spray formulation) etc. The composition is typically formulated as a liquid, solution, microemulsion, liposome, spray or other formulation type suitable for administration to a human subject, preferably the composition is formulated in a suitable dosage form for intranasal administration. In one embodiment preferably the composition is formulated as a liquid formulation or a spray formulation, suitable for intranasal administration.
5.

Antigen:

In one embodiment, the vaccine candidates produced are recombinant adenovirus vectors viz. rAd5-S, rAd5-S1, and adenovirus vector ChAd-S, which are used as antigen/antigenic component in the immunogenic composition or vaccine composition of invention.

Antigen Concentration:

Said “immunogenically effective concentration” of adenovirus vectors (antigenic vaccine candidates viz. rAd5-S, rAd5-S1, and ChAd-S) comprises a concentration in the range between 10\hat{ }9 to 10\hat{ }12 virus particles. In one embodiment the concentration of the above said adenovirus vector rAd5-S or rAd5-S1 or ChAd-S or combination thereof present in the composition and/or vaccine formulation and/or used in the method of treatment/prophylaxis of the present invention comprises a concentration selected from 10\hat{ }9, 10\hat{ }9.5, 10\hat{ }40, 10\hat{ }10.5, 10\hat{ }41, 10\hat{ }11.5, and 10\hat{ }12 virus particles or any other suitable value near to these falling with the above specified concentration range of 10\hat{ }9 to 10\hat{ }12 virus particles.

In one embodiment the concentration of adenovirus vector used is 10\hat{ }9.

In one embodiment the concentration of adenovirus vector used is 10\hat{ }9.5.

In one embodiment the concentration of adenovirus vector used is 10\hat{ }10.

In one embodiment the concentration of adenovirus vector used is 10\hat{ }10.5.

In one embodiment the concentration of adenovirus vector used is 10\hat{ }11.

In one embodiment the concentration of adenovirus vector used is 10\hat{ }11.5.

In one embodiment the concentration of adenovirus vector used is 10\hat{ }12.

Excipient(s):

The composition comprising one or more of adenovirus vectors (antigenic vaccine candidates viz. rAd5-S, rAd5-S1, and ChAd-S) of the present invention generally may comprise and/or formulated with or without one or more pharmaceutically acceptable excipient(s), suitable for vaccine composition or formulation to be administered in mammals through various routes of administration (specifically through intranasal) in suitable concentration, which may be selected from group comprising of buffer, cryo-protectant, salt or isotonic agent, stabilizer, diluent or carrier, propellant (for spray formulation) etc.

Buffer:

The composition may comprise and formulated with a buffer or mixture of two or more buffers. In one embodiment the buffer of the formulation composition may be selected from Tris buffer, phosphate buffer or combination/mixture thereof in a concentration range between 10 to 20 mM may be used. Tris-HCl at pH that ranges from 7.0 to 7.6 in a concentration range between 10 to 20 mM may be used. The above said concentration of buffer may be any wherein between the rage of 10 to 20 mM or may be 10 mM, 11 mM, 12 mM, 13 mM, 14Mm, 15 mM, 16 mM, 17 mM, 18 mM, 19 mM or 20 mM or near to any of these. In one preferred embodiment, Tris-HCl20 mM at pH that ranges from 7.0 to 7.6 is used.

Generally, the composition is formulated with buffer(s). A suitable buffer composition is prepared comprising one or more buffer+one or more following excipients:

Cryo-Protectant:

The compositions may generally comprise and formulated with a Cryo-protectant or mixture of two or more Cryo-protectants in a concentration range between 1.5 to 4.5%. In one embodiment the cryo-protectant used in the present invention is Glycerol present in a concentration between 1.5 to 4.5% or selected from below concentration values or near to any of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4% and 4.5%.

In one preferred embodiment concentration of Glycerol used is 1.5%.

In one preferred embodiment concentration of Glycerol used is 2.0%.

In one preferred embodiment concentration of Glycerol used is 2.5%.

In one preferred embodiment concentration of Glycerol used is 3.0%.

In one preferred embodiment concentration of Glycerol used is 3.5%.

In one preferred embodiment concentration of Glycerol used is 4.0%.

In one preferred embodiment concentration of Glycerol used is 4.5%.

In one most preferred embodiment 2.5% Glycerol is used in the composition.

Salt(s):

The compositions may generally comprise and formulated with one or more salt(s) selected from sodium chloride, magnesium chloride or potassium chloride which are used to provide and/or maintain osmolarity. In one embodiment the formulation comprises sodium chloride (NaCl) in the composition. In one embodiment, Sodium chloride for osmolarity in a concentration range between 20 to 30 mM is used. In one embodiment the concentration of salt used is selected from below concentration values or near to any of 20 mM, 21 mM, 22 mM, 23 mM, 24 mM, 25 mM, 26 mM, 27 mM, 28 mM, 29 mM or 30 mM. In one preferred embodiment Sodium chloride 20 mM or 25 mM or 30 mM is used. In a most preferable embodiment 30 mM Sodium chloride is used in the composition for osmolarity. In another most preferable embodiment 25 mM Sodium chloride is used in the composition for osmolarity.

Stabilizer(s):

The vaccine composition and formulation may also comprise one or more suitable stabilizer(s) in an appropriate concentration to impart stability to the vaccine composition and/or formulation. Examples of such suitable stabilizer(s) that may be included in the vaccine composition comprises Magnesium chloride (MgCl), Polysorbate-80 (P-80) or like. Other stabilizer suitable for nasal vaccine may also be considered.

In one embodiment Magnesium chloride is used as a stabilizer in a concentration range between 1 to 4 mM. In one embodiment the concentration of Magnesium chloride used is selected from below concentration values or near to any of 1 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM. In one preferred embodiment Magnesium chloride 1 mM or 2 mM or 3 mM or 4 mM is used. In a most preferable embodiment 2 mM Magnesium chloride is used in the composition.

In one embodiment Polysorbate-80 is used as stabilizer in a concentration range between 0.01 to 0.2%. In one embodiment use of polysorbate in the composition is optional. When used in the composition, it may be present in a concentration selected from 0.01%, 0.02%, 0.03%, 0.04%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19% and 0.2%.

In one embodiment 0.1% Polysorbate-80 is used in the composition.

In another embodiment 0.2% Polysorbate-80 is used in the composition.

In one embodiment Magnesium chloride alone is used. In one embodiment Polysorbate-80 alone is used. In one embodiment the composition may comprise both Magnesium chloride and Polysorbate-80 within the concentration range specified above. In one most preferred embodiment, the composition of the present invention comprises Magnesium chloride 2 mM, and Polysorbate-80 at 0.1%.

Diluent:

The composition of the formulation may generally comprise a pharmaceutically acceptable diluent or carrier which is non-toxic and is compatible with the viral vector antigen of the present invention. The diluent or carrier may be an inert solvent or a medium for liquid solution or dispersion which may be selected from water, polyol (for example, glycerol, and the like), and suitable mixtures thereof.

In certain embodiment, the formulation may comprise a surfactant such as an ionic surfactant or non-ionic surfactant depending upon the type of dosage form and delivery mode selected. In one embodiment the invention uses polysorbate, preferably polysorbate 80. The use of surfactant or polysorbate is optional.

In one embodiment, the vaccine composition and/or the immunogenic composition comprises following ingredients:
Antigen: active ingredient adenovirus vector selected from rAd5-S or rAd5-S1 or ChAd36 or any combination thereof as antigen(s) in a concentration range between 10\hat{ }9 to 10\hat{ }12 virus particles;
Buffer: buffered with Tris-HCl or phosphate buffer or combination thereof at pH that ranges from 7.0 to 7.6 in a concentration range between 10 to 20 mM,
Cryo-protectant: Glycerol in a concentration range between 1.5 to 4.5%,
Salt: Sodium chloride for osmolarity in a concentration range between 20 to 30 mM,
Stabilizer: Magnesium chloride in a concentration range between 1 to 4 mM, and
Stabilizer (optional): Polysorbate-80 in a concentration range between 0.01 to 0.2%.

In one preferred embodiment, the vaccine composition and/or the immunogenic composition comprises following ingredients:
Antigen: active ingredient adenovirus vector selected from rAd5-S or rAd5-S1 or ChAd36 or any combination thereof as antigen(s) in a concentration range between 10\hat{ }9 to 10\hat{ }12 virus particles;
Tris-HCl 20 mM at pH that ranges from 7.0 to 7.6,
Glycerol 2.5%,
Sodium chloride 25 mM,
Magnesium chloride 2 mM, and
Polysorbate-80 at 0.1%.

A Stable immunogenic composition, wherein the one or more active ingredient adenovirus vector(s) between 10\hat{ }9 to 10′42 virus particles as antigen(s) are buffered with Tris-HCl at pH that ranges from 7.0 to 7.6 in a concentration range between 10 to 20 mM, Glycerol as cryo-protectant in a concentration range between 1.5 to 4.5%, Sodium chloride for osmolarity in a concentration range between 20 to 30 mM, Magnesium chloride as stabilizer in a concentration range between 1 to 4 mM, and with or without Polysorbate-80 as stabilizer in a concentration range between 0.01 to 0.2%.

In one embodiment, the vaccine composition and formulation of the present invention typically will be sterile.

In one embodiment, the vaccine composition and/or formulation of present invention is stable under manufacture and/or storage condition. In one embodiment, the vaccine formulation maintains the desired antigenic titer to elicit desired immunogenic response. In one embodiment, the vaccine formulation is stable at the storage condition of 2-8° C.

The composition of the present invention typically may be formulated in a suitable dosage form. In one embodiment the composition may be formulated as a liquid preparation such as solution, suspension, emulsion or microemulsion, which may be administered in drop(s) or may be formulated as a spray formulation or other formulations, wherein said preparation/formulation being suitable for administration to a human subject, more preferably, the composition is formulated in a suitable form for intranasal administration. In one embodiment the vaccine of the present invention is nasal vaccine and to deliver nasal vaccine various suitable form of formulation may be selected such as nasal drops, sprays and the like. It may also be formulated as nasal gel, aerosol. In one preferred embodiment, the composition of the present invention as described above is formulated as a liquid formulation such as liquid drop(s), suitable for intranasal administration.

In another preferred embodiment, the composition of the present invention is formulated in a suitable dosage form suitable for nasal administration such as nasal inhalation for example spray formulation or like suitable for intranasal administration. When such spray is formulated, one or more suitable excipients such as propellant may be added in the formulation composition to make the formulation sprayable or aerosol type.

In one embodiment the drug substance (antigen) i.e. the recombinant adenovirus vector (rAd5-S or rAd5-S1 or ChAd-S) in a concentration between 10\hat{ }9 to 10\hat{ }12 virus particles is diluted with formulation buffer as mentioned in example 16 and a vaccine formulation is obtained. In one embodiment rAd5-S or rAd5-S1 or ChAd-S is formulated with formulation buffer as mentioned in Example 16, with or without polysorbate-80 elicited immune response against spike protein.

Such composition is provided as a final vaccine formulation or further formulated in a suitable dosage form as described above to give the final vaccine formulation. The vaccine formulation may be stored in temperature range of 2-8° C. The immunogenic composition as described above can be injected intranasally.

Combination Vaccine

The immunogenic composition as described above can be injected intranasally, optionally in combination with one or more other immunogenic composition. The combination vaccine if prepared, it may be prepared by combining immunogenic composition of present invention with one or more other immunogenic composition(s), wherein the said immunogenic compositions may be provided either in the same composition (single composition) or in two separate compositions in two separate containers, which may be administered to a subject under treatment simultaneously or concurrently. Exemplary of said “one or more other immunogenic composition(s)” may be immunogenic compositions for various types of live or replication-defective Influenza vaccines.

The immunogenic composition as described above can be administered in a prime-boost regimen where any vaccine against COVID, including live attenuated, killed, vectored, recombinant, DNA, subunit or other vaccine can be administered first followed by booster immunizations with the claimed composition, or vice versa, and repeated in any sequence for as long as required to develop robust immunity in the host species being targeted. The immunogenic composition as described above does not induce antibodies to non-structural proteins of SARS-CoV-2, so that tests designed to differentiate vaccinated and infected humans based on the detection of antibodies to certain non-structural proteins can be employed in combination with immunization with the claimed composition.

In one embodiment the present invention describes a stable composition which is immunogenic in multiple routes of administration including intranasal and intramuscular administration. Examples 18, 19, 20, 21 and 22 provides examples of intranasal (I/N) and intramuscular (UM) administration.

The stable formulation of the adenovirus vector (rAd5-S or rAd5-S1 or ChAd-S) as described in the Example 16, which will elicit immune response against the spike protein. The present invention provides a stable vaccine composition comprising or expressing one or more of the antigen (partial, chimeric, designer, RBD alone or complete spike glycoprotein (S) or subunit (S1) of the SARS-CoV-2) in various systems, the said antigens being formulated with or without one or more pharmaceutically acceptable excipient(s) as described above and further in examples, wherein the vaccine composition elicits protective immune response to SARS-CoV-2 in mammals when administered through intranasal, oral, intramuscular routes.

| S. No. | Ingredients | Concentration |
|---|---|---|
| | Antigen | |
| 1. | Adenovirus vector | between 10\hat{ }9 to 10\hat{ }12 |
| | (rAd5-S or rAd5-S1 or ChAd-S) | virus particles |
| | Buffer Composition | |
| 2. | Tris-HCl (pH 7.0 to 7.6) | 10 to 20 mM |
| 3. | Glycerol | 1.5 to 4.5% |
| 4. | Sodium Chloride | 20 to 30 mM |
| 5. | Magnesium Chloride | 1 to 4 mM |
| 6. | Polysorbate-80 (optional) | 0.01 to 0.2% |

The above table represents a general composition of the present invention which comprises antigen (Adenovirus vector, vaccine candidate)+Buffer with concentration range of each ingredient. Specific immunogenic composition or vaccine composition formulated as vaccine which is used and/or tested for animal study of the present invention (Examples 18-24) is further described in Example-16.

Method of Treatment

In another aspect the invention discloses a method of treatment and/or prophylaxis for COVID-19 and/or eliciting an immune response against SARS-CoV-2 and its variants in mammals including human subjects, wherein the said method comprises nasal administration of a vaccine formulation comprising: a vaccine composition or a stable immunogenic composition as described above and exemplified below.

In one preferred embodiment, the said method of treatment and/or prophylaxis for COVID-19 and/or eliciting an immune response against SARS-CoV-2 comprises intranasal administration of novel vectors as antigen in a dose range between 10\hat{ }9 to 10\hat{ }12 virus particles, which novel vectors are produced by using below three different spike protein constructs 1-3 (respectively presented in SEQ. ID No. 1-3) through human adenovirus type 5 (Ad5) or Chimpanzee adenovirus 36 (ChAd36)” as a vaccine candidate, viz.

Vector Construct-1:
Ad5 expresses full-length (S) SARS-CoV-2 spike protein (SARS-CoV2-S),

Vector Construct-2:
Ad5 expresses subunit (51) of SARS-CoV-2 spike protein (SARS-CoV2-S1),

Vector Construct-3:
ChAd36 expresses full-length (S) SARS-CoV-2 spike protein (SARS-CoV2-S).

Optionally, in the above said method of treatment, the above said vaccine composition/formulation may be administered in combination with other suitable immunogenic composition which may be selected from immunogenic compositions for various types of live or replication-defective Influenza vaccine compositions.

The vaccine composition, immunogenic composition and method of treatment as described above, wherein the composition or vaccine is formulated in a suitable dosage form, stored in a suitable container suitable for nasal or intranasal application to human subjects in a suitable dose sufficient to elicit an immune response against SARS-CoV-2 and its variants in mammals. The dosage form comprises liquid of drop(s) or spray form or other similar forms suitable for intranasal administration or suitable for administration to a human subject for the treatment of COVID-19 and SARS-CoV-2 infection. In one embodiment the above said suitable dose of adenovirus vector or formulated composition comprises between 10\hat{ }9 to 10\hat{ }12 virus particles.

The SARS-CoV-2 antigen compositions comprising partial, chimeric or complete spike glycoprotein or subunit S1, more particularly comprising rAd5-S or rAd5-S1 or ChAd-S is effective against any betacoronavirus strains that share anywhere between 50% to 100% identity at any other region of the genome particularly in the amino acid levels in the receptor binding domain of the spike glycoprotein.

Method of Estimation of Infectious Units

In one aspect, the invention describes a method to estimate the infectious unit by detection of the transgene (spike) expression by the adenovirus vector by chromogenic substrate but not limited to 3-Amino-9-Ethylcarbazole (AEC) or 3, 3′-Diaminobenzidine (DAB) that correlates to the expression of the transgene (spike protein) which can be extrapolated to the detection of the quantity of spike expressing units in the sample.

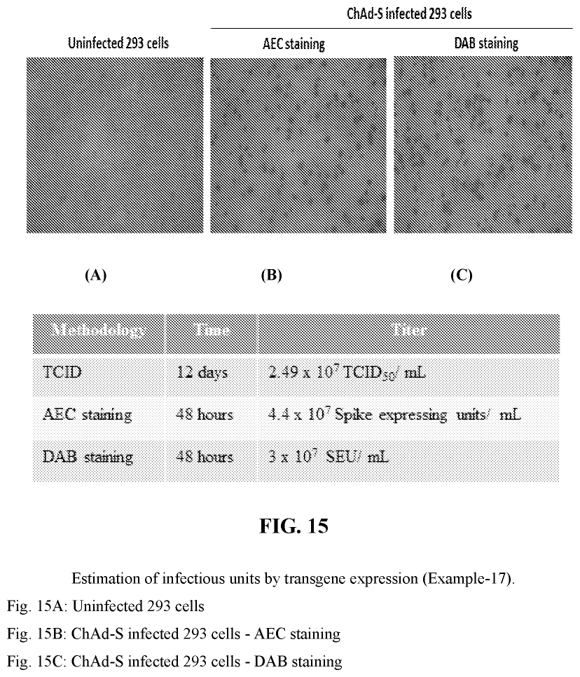

The adenovirus vector infectious units are estimated with conventional tissue culture infectious dose 50 or plaque forming units (pfu), which usually takes 10 to 12 days. In one aspect, the present invention describes a method to estimate the infectious units by detection of the transgene expression. As mentioned in Example 17 and shown in FIG. 15, this method allows the estimation of the infectious units within 48 hours. In the said method of estimation spots developed by any chromogenic substrates are correlated to the expression of the transgene. distinct spots developed by any chromogenic substrates such as 3-Amino-9-Ethylcarbazole (AEC) or 3,3′-Diaminobenzidine (DAB) that correlates to the expression of the transgene (spike protein) which can be extrapolated to the detection of the quantity of spike expressing units in the sample.

### EXAMPLES

The above describe aspects of the invention further be understood by following non-limiting examples 1-24 and corresponding drawing FIGS. 1-22.

### Example-1: Diagrammatic Representation of SARS-CoV-2 Genome as Shown in FIG. 1

The SARS-CoV-2 genome is a linear, positive sense single stranded RNA of about −29000 nucleotides. Like other coronaviruses, SARS-CoV-2 has four structural proteins, known as the S (spike), E (envelope), M (membrane), and N (nucleocapsid) proteins. The spike protein is surface-exposed and mediates entry into host cells, spike is the main target of neutralizing antibodies upon infection and the focus of vaccine design.

### Example-2: Representative Nucleotide Sequence of Spike Region of SARS-CoV-2 Genome

The nucleotide sequences of SARS-CoV-2 spike region synthetic constructs. The present invention uses three constructs, the sequence listing of which are in SEQ. ID. No. 1, SEQ. ID. No. 2, and SEQ. ID. No. 3.

SEQ. ID. NO. 1:

Nucleotide sequence of SARS-CoV-2 spike region synthetic construct 1: SARS-CoV2-S.

SEQ. ID. NO. 2:

Nucleotide sequence of SARS-CoV-2 spike region synthetic construct 2: SARS-CoV2-S1.

SEQ. ID. NO. 3:

Nucleotide sequence of SARS-CoV-2 spike region synthetic construct 3: SARS-CoV2-S.

### Example-3: Schematic Representation of the Different Spike Gene Constructs Incorporated in the Adenovirus as Shown in FIG. 2

The adenovirus vectors with the synthetic spike gene or part of spike gene with the representative promoters (MCMV, CMV), control elements (Tet-O or Lac-O) are depicted in the image of FIG. 2.

Construct-1 and its Production/Preparation Procedure:

The transgene human codon optimized spike gene of SARS-CoV2 (SARS-CoV2 S) was cloned into NheI and SalI restriction sites. The transgene expression is driven by the murine cytomegalovirus promoter (pMCMV) which is controlled by the presence of the lac operator (Lac-O). An intron sequence is introduced between the Lac-O and the transgene. Downstream of the transgene, a poly A tail and Frt site was introduced for better transcription of the transgene and recombination with the adenovirus genomic plasmid, respectively. A pictorial representation of the recombination is depicted in FIG. 4.

Construct-2 and its Production/Preparation Procedure:

The transgene human codon optimized S1 region of spike gene of SARS-CoV2 (SARS-CoV2 S1) was cloned into NheI and SalI restriction sites. The transgene expression is driven by the murine cytomegalovirus promoter (pMCMV) which is controlled by the presence of the lac operator (LacO). An intron sequence is introduced between the Lac-O and the transgene. Downstream of the transgene, a poly A tail and Frt site was introduced for better transcription of the transgene and recombination with the adenovirus genomic plasmid, respectively. A pictorial representation of the recombination is depicted in FIG. 4.

Construct-3 and its Production/Preparation Procedure:

The transgene pre-fusion stabilized spike gene of SARS-CoV2 or its variants (SARS-CoV2 S) were driven by the cytomegalovirus promoter (CMV) which is controlled by the presence of the two tet operator (2Tet-O). Downstream of the transgene, a poly A tail was introduced for better transcription and translation.

### Example-4: Schematic Representation of the Control Elements to Regulate Protein Expression Via the Recombinant Adenovirus as Shown in FIG. 3 (www.microbix.com)

AdMax Hi-IQ system was obtained from Microbix Bioscience Inc., Canada for generation of replication-defective recombinant adenovirus. The SARS-CoV-2 spike genes are under the control of the bacterial lac operator and the 293 cells express the lac repressor so that the recombinant gene is not active during the generation of Ad vectors as the repressor binds to the operator and does not allow transcription. On the other hand, in any other cells where the repressor is absent, the proteins are expressed, and allows the generation of high adenoviruses with high level of viral protein expression.

Similar control of transgene expression is expected if tet operator (tet-o) controls the CMV promoter in presence of tet-repressor.

### Example-5: Schematic Representation of the Ad-Max HiIQ System of Generation of Recombinant Adenoviruses as Shown in FIG. 4 (www.microbix.com)

The SARS-CoV-2 spike constructs (i.e., foreign DNA) are cloned into the shuttle plasmid, which is recombined with the genomic plasmid in 293 cells through co-transfection. The plasmids contain loxP sites for recombination mediated by the FLP enzyme, which is expressed from the genomic plasmid. Recombination produces a full-length adenovirus with SARS-CoV-2 spike gene inserted in place of the E1 region. Proteins encoded in the E1 region are essential for replication of adenoviruses, and this function is provided in trans by 293 cells, which carry a part of adenoviral genome including the E1 region. Similar control of transgene expression is expected if tet operator (tet-o) controls the CMV promoter.

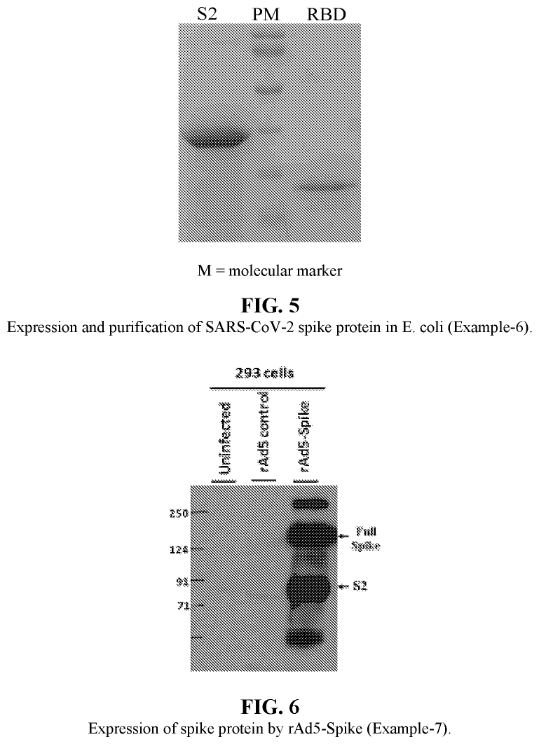

### Example-6: Expression and Purification of SARS-CoV-2 Spike Protein in E. coli as Shown in FIG. 5

SARS-CoV-2 spike genes coding for RBD and S2 were cloned in bacterial expression vectors along with hexahistidine tag under the lac operon control system, and the expression of recombinant proteins was induced by using isopropyl □-D-1-thiogalacto-pyranoside (IPTG). Bacterial lysates were applied to metal affinity chromatography columns containing Ni-NTA resin to purify the proteins. The purity of the proteins was assessed by SDS-PAGE and the authenticity was confirmed by Western blotting using appropriate antibodies (not shown). These proteins were used as antigens for raising polyclonal sera, which were used to assess protein expression by the recombinant adenovirus vectors as well as in assays to evaluate antibody and T cell responses.

Expression of Spike Protein by rAd5

### Example-7: Expression of Spike Protein by rAd5-Spike

The 293 cells were infected with 5 MOI with rAd5 control or rAd5-Spike or were left uninfected. Forty-eight hours post infection, the cells were scrapped and lysed with lysis buffer (50 mM Tris-HCl [pH 7.5], 100 mM NaCl, 1% (3-((3-cholamidopropyl) dimethyl ammonio)-1-propanesulfonate (CHAPS), and 1 mM phenylmethylsulfonyl fluoride). The lysates from uninfected 293 cells and infected 293 cells (rAd5 control, rAd5-spike) were loaded on 10% SDS-PAGE and western blot with S2 antibody. Since, S2 antibody was used for the detection of spike expression, both full length spike and S2 were specifically in the rAd5-Spike lane. The result is depicted and shown in FIG. 6.

### Example-8: Expression of S1 by rAd5-S1

The 293 cells were infected with 5 MOI with rAd5 control or rAd5-S1 or were left uninfected. Forty-eight hours post infection, the cells were scrapped and lysed with lysis buffer (50 mM Tris-HCl [pH 7.5], 100 mM NaCl, 1% (3-((3-cholamidopropyl) dimethyl ammonio)-1-propanesulfonate (CHAPS), and 1 mM phenylmethylsulfonyl fluoride). The lysates from uninfected 293 cells and infected 293 cells (rAd5 control, rAd5-S1) were loaded on 10% SDS-PAGE and western blot with S1 antibody. The presence of specific S1 protein in the third lane confirmed the expression of S1 fragment of spike protein by rAd5-S1. The result is depicted and shown in FIG. 7.

### Example-9: Expression of Spike Protein by ChAd-S

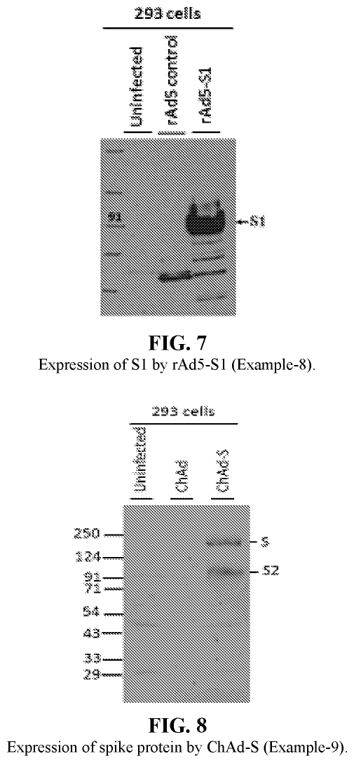

The 293 cells were infected with 5 MOI with ChAd or ChAd-S or were left uninfected. Forty-eight hours post infection, the cells were scrapped and lysed with lysis buffer (50 mM Tris-HCl [pH 7.5], 100 mM NaCl, 1% (3-((3-cholamidopropyl) dimethyl ammonio)-1-propanesulfonate (CHAPS), and 1 mM phenylmethylsulfonyl fluoride). The lysates from uninfected 293 cells and infected 293 cells (ChAd control, ChAd-S) were loaded on 10% SDS-PAGE and western blot with S2 antibody confirmed the expression of full-length spike and S2 protein by ChAd-S. The result is depicted and shown in FIG. 8.

Growth Kinetics Characterization

### Example-10: Growth Kinetics of rAd5-S

Cells were infected with rAd5-S at 0.001, 0.003, 0.01, 0.05, 0.1 and 0.5 MOI's (multiplicity of infection). Harvests were analyzed to estimate the rAd5-S tissue culture infectious dose 50/mL at 48, 72, 96, 120, 144 and 168 hours post infection and the log 10 infectious titers were plotted An optimum titer was obtained with 0.5 MOI infection between 96 to 120 hours. The result is depicted and shown in FIG. 9.

### Example-11: Growth Kinetics of ChAd-S

Cells were infected with Chimpanzee adenovirus 36 expressing SARS-CoV2 pre-fusion stabilized spike protein at 0.25, 0.5, 1, 2, 3 MOI's. Harvests were analyzed to estimate the ChAd-S tissue culture infectious dose 50/mL at 12, 24, 36, 48, 60, 72, 84 and 96 hours and the log 10 infectious titers were plotted. Optimum titer was obtained with 1 MOI of ChAd-S infection at 60 hours post infection. The result is depicted and shown in FIG. 10.

Process Workflow for Manufacture

### Example-12: Process Workflow for Manufacture of Adenovirus Vectored COVID-19 Vaccine

The manufacturing process for the production of the adenovirus vectored COVID-19 vaccine candidate is mentioned in the flow diagram FIG. 11.

The harvests were lysed, clarified and sterile filtered with depth filters. These samples were either purified with anion exchange chromatography (FIGS. 13 and 14) or concentrated between 10-15 times. The concentrates were purified with size exclusion chromatography which was followed by approximately 3 to 5 times concentration to produce the drug substance and it is stored at 2-8° C. The drug substance is diluted with formulation buffer as mentioned in example 16. As shown in FIG. 12 workflow, the process comprises following steps:

Upstream Production:

The 293 cells were infected with 1 MOI of rAd5-S or rAd5S1 or ChAd-S and incubated at 37±1° C. for 3-4 days.

Cell Lysis and DNA Fragmentation:

The infected cells were harvested by lysis with buffer consisting of 0.1% TritonX-100, 0.05% polysorbate 80 with DNA fragmentation enzymes, such as benzonase or DNA precipitation.

Clarification:

Clarification was performed wherein the debried were removed from the lysates with 5 micron and 0.8 micron filters.

Concentration, Purification and Buffer Exchange:

These steps involve—the tangential flow filtration (TFF) with 750 KDa cut-off was utilized for the concentration of the clarified antigen up to 10-15 times. The concentrated samples where purified by size-exclusion chromatography and the flow-through is subjected to buffer exchange with the formulation buffer with TFF.

Sterile Filtration:

The step of sterile filtration is performed with 0.2 micron filters.

Drug Substance Storage:

The drug substance i.e. vaccine candidate prepared is stored at 2-8° C. until formulation.

Formulation Storage:

The drug substance is diluted with formulation buffer and further stored at 2-8° C.

### Example-13: Lysis Methods and Adenovirus Recovery

The 293 cells were infected with rAd5-Spike with 1 MOI. The infected cells were scrapped after 60 hours post infection. Post-infection the cells infected with adenovirus vector were lysed with five different methods and the recovered virus titer is mentioned. The results (FIG. 12) indicated that the virus recovery with buffer consisting of 0.1% TritonX-100, 0.05% polysorbate 80 was the maximum.

### Example-14: Anion-Exchange Chromatography of rAd5-Spike

The clarified cell lysates were purified by anion-exchange chromatography, samples were loaded on 10% SDS-PAGE and the stained with coomassie. Representative images of rAd5-Spike (FIG. 13, A and B) purification are provided as an example.

The anion exchange chromatography columns were equilibrated with five column volumes of equilibration buffer (0.1 M NaCl, 50 mM Tris (pH 8.0) and 5% glycerol). The rAd5-Spike preparations were mixed with equilibration buffer at a ratio of 1:7 and loaded on to the column gently without disturbing the beads, followed by passing five column volumes of elution buffer with increasing concentration of NaCl (0.2 M to 0.7 M NaCl, 50 mM Tris (pH 8.0) and 5% glycerol). The eluted fractions were collected and the absorbance was measured at 280 nm. The elution buffer was not changed until the absorbance of the eluate at 280 nm reached zero. After the elution buffer containing 0.7 M NaCl was passed through the column, the column was washed with 200 to 300 mL of 0.1 N NaOH. The column was washed extensively with sterile water, until the eluate attained a pH of 7.0. The fractions were subjected to SDS-PAGE and stained with Coomassie brilliant blue to analyze the purity. The elution buffer with 0.4 M NaCl yielded the purest form of adenovirus.

### Example-15: Anion-Exchange Chromatography of ChAd-S

The clarified cell lysates were purified by anion-exchange chromatography, samples were loaded on 10% SDS-PAGE and the stained with coomassie. Representative images of ChAd-S (FIG. 14, A and B) purification are provided as an example.

The anion exchange chromatography columns were equilibrated with five column volumes of equilibration buffer (0.1 M NaCl, 50 mM Tris (pH 8.0) and 5% glycerol). The ChAd-S preparations were mixed with equilibration buffer at a ratio of 1:7 and loaded on to the column gently without disturbing the beads, followed by passing five column volumes of elution buffer with increasing concentration of NaCl (0.2 M to 0.7 M NaCl, 50 mM Tris (pH 8.0) and 5% glycerol). The eluted fractions were collected and the absorbance was measured at 280 nm. The elution buffer was not changed until the absorbance of the eluate at 280 nm reached zero. After the elution buffer containing 0.7 M NaCl was passed through the column, the column was washed with 200 to 300 mL of 0.1 N NaOH. The column was washed extensively with sterile water, until the eluate attained a pH of 7.0. The fractions were subjected to SDS-PAGE and stained with Coomassie brilliant blue to analyze the purity. The elution buffer with 0.4 M NaCl yielded the purest form of adenovirus.

### Example-16: Formulation of Adenovirus Vector(s)

The composition of stable formulation of adenovirus vector for administration in mammals may be prepared based on below shown general composition.

Example 16A: General formulation Composition

| S. No. | Ingredients | Concentration |
|---|---|---|
| 1. | Adenovirus vector | between 10\hat{ }9 to 10\hat{ }12 |
| | rAd5-S or rAd5-S1 or ChAd-S | virus particles |
| 2. | Tris-HCl (pH 7.0 to 7.6) | 10 to 20 mM |
| 3. | Glycerol | 1.5 to 4.5% |
| 4. | Sodium Chloride | 20 to 30 mM |
| 5. | Magnesium Chloride | 1 to 4 mM |
| 6. | Polysorbate-80 (optional) | 0.01 to 0.2% |

### Example 16A

Prepared a mixture of ingredients consisting of Tris-HCl (pH 7.0 to 7.6) in a concentration selected between 10 to 20 mM, Glycerol in a concentration selected between 1.5 to 4.5%, Sodium Chloride in a concentration selected between 20 to 30 mM, Magnesium Chloride in a concentration selected between 1 to 4 mM, Polysorbate-80 in a concentration selected between 0.01 to 0.2%. This mixture was sterile filtered and the sterile preparation of rAd5-S or rAd5-S1 or ChAd-S in a concentration selected between 10\hat{ }9 to 10\hat{ }12 virus particles were mixed and stored at 2-8° C.

Example 16B: Specific Formulation-with rAd5-S

| | Tris- | | Sodium | Magnesium | Polysor- |
|---|---|---|---|---|---|
| rAd5-S | HCl | Glycerol | Chloride | Chloride | bate-80 |
| 10\hat{ }9 to 10\hat{ }12 | 20 mM | 2.5% | 25 mM | 2 mM | 0.1% |

### Example 16B

Prepared a mixture of ingredients consisting of Tris-HCl (pH 7.0 to 7.6) 20 mM, Glycerol 2.5%, Sodium Chloride 25 mM, Magnesium Chloride 2 mM, Polysorbate-80 0.1%. This mixture was sterile filtered and the sterile preparation of rAd5-S between 10\hat{ }9 to 10\hat{ }12 virus particles were mixed and stored at 2-8° C.

Example 16C: Specific Formulation-with rAd5-S1

| | Tris- | | Sodium | Magnesium | Polysor- |
|---|---|---|---|---|---|
| rAd5-S1 | HCl | Glycerol | Chloride | Chloride | bate-80 |
| 10\hat{ }9 to 10\hat{ }12 | 20 mM | 2.5% | 25 mM | 2 mM | 0.1% |

### Example 16C

Prepared a mixture of ingredients consisting of Tris-HCl (pH 7.0 to 7.6) 20 mM, Glycerol 2.5%, Sodium Chloride 25 mM, Magnesium Chloride 2 mM, Polysorbate-80 0.1%. This mixture was sterile filtered and the sterile preparation of rAd5-S1 between 10\hat{ }9 to 10\hat{ }12 virus particles were mixed and stored at 2-8° C.

Example 16D: Specific Formulation-with ChAd-S

| | Tris- | | Sodium | Magnesium | Polysor- |
|---|---|---|---|---|---|
| ChAd-S | HCl | Glycerol | Chloride | Chloride | bate-80 |
| 10\hat{ }9 to 10\hat{ }12 | 20 mM | 2.5% | 25 mM | 2 mM | 0.1% |

### Example 16D

Prepared a mixture of ingredients consisting of Tris-HCl (pH 7.0 to 7.6) 20 mM, Glycerol 2.5%, Sodium Chloride 25 mM, Magnesium Chloride 2 mM, Polysorbate-80 0.1%. This mixture was sterile filtered and the sterile preparation of ChAd-S between 10\hat{ }9 to 10\hat{ }12 virus particles were mixed and stored at 2-8° C.

Following the same procedure as described above for 16A-16D, other similar formulations for each of rAd5-S or rAd5-S1 or ChAd-S taking various concentrations of each ingredients within the concentration range mentioned in the general formulation composition Example-16A may be prepared.

### Example-17: Estimation of Infectious Units by Transgene Expression

The ChAd-S infected cells and uninfected 293 cells, 48 hours post infection, the cells were fixed and probed with rabbit polyclonal S1 antibody followed by anti-rabbit IgG peroxidase. The peroxidase activity was detected by 3-Amino-9-Ethylcarbazole (AEC) or 3,3′-Diaminobenzidine (DAB). Comparative titers of ChAd-S infectious units as estimated by classical TCID, AEC staining and DAB staining is provided. The results indicate that both AEC and DAB staining can be used to detect the spike expression and count the number of spike expressing units of ChAd in the sample. (FIG. 15)

### Example-18: Serum IgG Antibody Response of Mice Immunized with rAd5-Spike

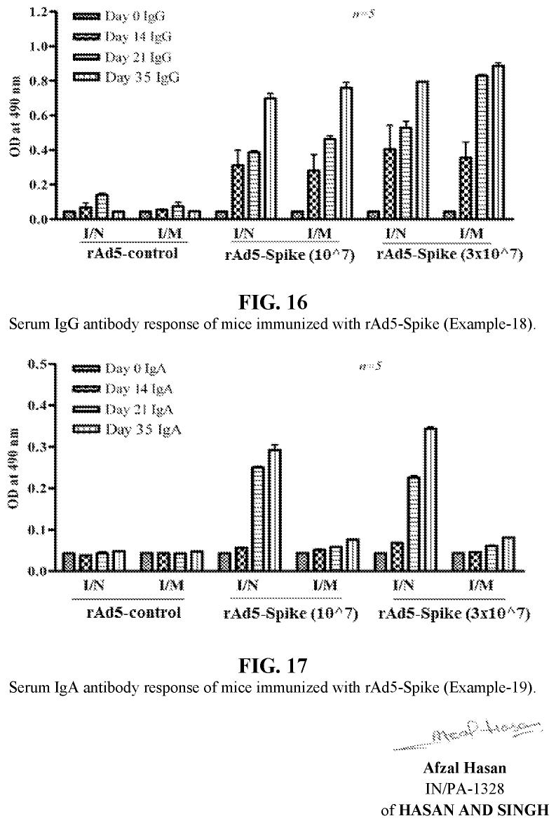

The serum anti-S1 IgG antibody responses of mice immunized through intranasal or intramuscular route with rAd5-control or rAd5-Spike are depicted as bar diagrams. The results indicate that there is a dose-dependent increase in the IgG antibody against S1, when the animals were immunized through intranasal route or intramuscular route with rAd5-Spike. (FIG. 16)

### Example-19: Serum IgA Antibody Response of Mice Immunized with rAd5-Spike

The serum anti-S1 IgA antibody responses of mice immunized through intranasal or intramuscular route with rAd5-control or rAd5-Spike are depicted as bar diagrams. The results indicate that there is a dose-dependent response of serum anti-S1 IgA antibody only in animals that were immunized with rAd5-Spike through intranasal route. Intramuscular immunization did not elicit IgA response in the animals. (FIG. 17)

### Example-20: Bronchio-Alveolar IgA Antibody Response of Mice Immunized with rAd5-Spike

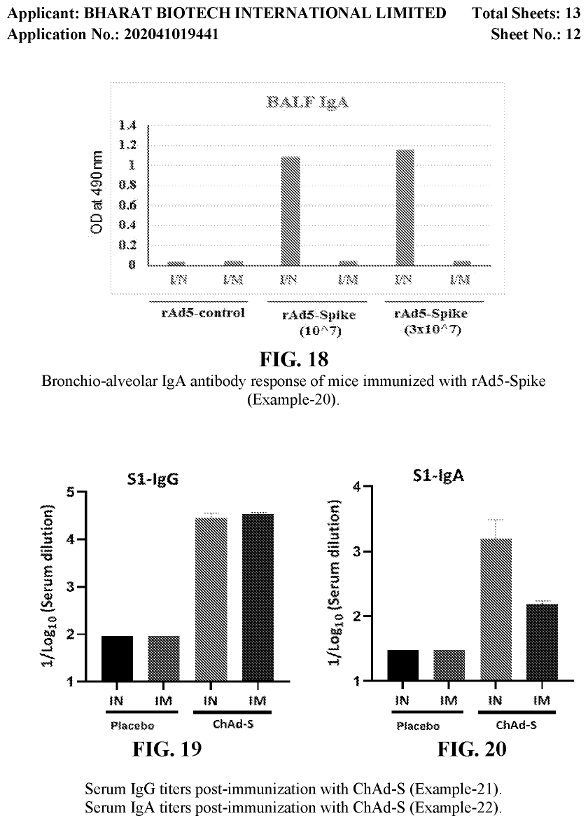

The IgA antibody responses in bronchio-alveolar lavage fluid samples from mice immunized through intranasal or intramuscular route with rAd5-control or rAd5-Spike are depicted as bar diagrams. The bronchio-alveolar lavage fluid samples consisted of anti-S1 IgA antibody only in animals that were immunized through intranasal route with rAd5-Spike. Intramuscular immunization did not elicit IgA response in the animals. (FIG. 18)

### Example-21: Serum IgG Titers Post-Immunization with ChAd-S

The serum anti-S1 IgG antibody titers of mice immunized with ChAd-S through intranasal and intramuscular routes are depicted as bar diagrams. The serum anti-S1 IgG levels in animals immunized through both intramuscular and intranasal route of administration were significantly higher compared to the animals that were administered with placebo. (FIG. 19)

### Example-22: Serum IgA Titers Post-Immunization with ChAd-S

The serum anti-S1 IgA antibody titers of mice immunized with ChAd-S through intranasal and intramuscular routes are depicted as bar diagrams. Serum IgA response was specifically observed only in animals administered with ChAd-S through intranasal route. (FIG. 20)

### Example-23: Serum IgG Antibody Response of Mice Immunized with ChAd-S Vector

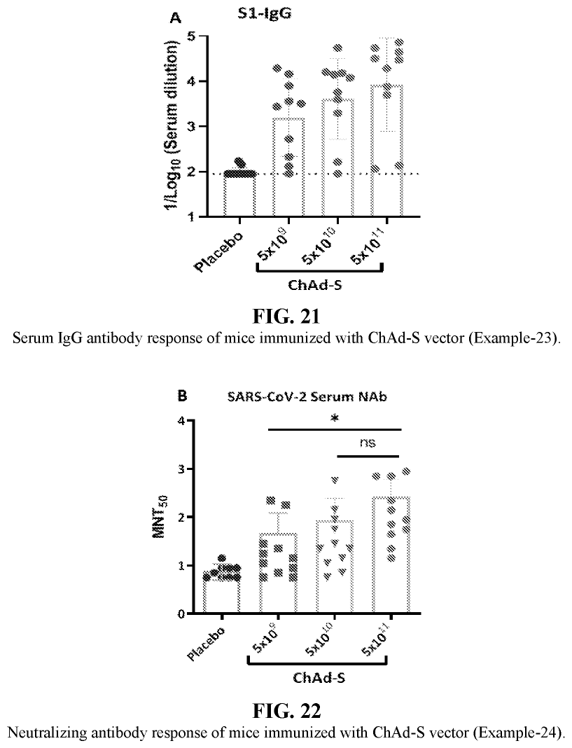

The serum anti-S1 IgG titers of mice immunized with three different doses of ChAd-S through intranasal route is provided as an example. Dose-dependent increase in serum anti-S1 IgG titer was observed in animals that were administered with ChAd-S through intranasal route. (FIG. 21)

### Example-24: Neutralizing Antibody Response of Mice Immunized with ChAd-S Vector

The SARS-CoV2 neutralizing antibody titers of mice immunized with three different doses of ChAd-S through intranasal route is provided as an example. Dose-dependent increase in the SARS-CoV2 neutralization titer was observed with the mice administered with ChAd-S vector. The highest neutralizing antibody response was observed with the highest dose of ChAd-S. (FIG. 22)

The compositions as described above, and vaccine provided in the present invention is effective against any betacoronavirus strains that share anywhere between 50% to 100% identity at any other region of the genome particularly in the amino acid levels in the receptor binding domain of the spike glycoprotein.

We claim:

1. A coronavirus vaccine composition for prophylaxis and eliciting an immune response against SARS-CoV-2 and its variants in mammals, the said vaccine composition comprises an immunogenic composition comprising one or more viral vector(s) engineered to express SARS-CoV-2 spike protein.

2. The vaccine composition as claimed in claim 1, wherein the vaccine is adenovirus vectored vaccine for Covid-19, wherein the viral vector used is adenovirus.

3. The vaccine composition as claimed in claim 2, wherein said adenovirus viral vector used in the expression system may be Human adenovirus, Chimpanzee adenovirus or other suitable adenovirus species.

4. The vaccine composition as claimed in claim 3, wherein the adenovirus is human adenovirus type-5 (Ad5) or Chimpanzee adenovirus 36 (ChAd36).

5. The vaccine composition as claimed in claim 4, wherein the adenovirus type-5 (Ad5) or Chimpanzee adenovirus 36 (ChAd36) is engineered to express SARS-CoV-2 spike glycoprotein.

6. The vaccine composition as claimed in claims 2-5, wherein the said use of adenovirus vector is to express spike glycoprotein of SARS-CoV-2 and its variants in mammals.

7. The vaccine composition as claimed in claims 5-6, wherein the said spike glycoprotein of SARS-CoV-2 which is expressed in adenovirus may be full length spike or parts thereof including RBD alone or designer or truncated or partial or chimeric spike or S1 subunit or S2 subunit.

8. The vaccine composition as claimed in claim 7, wherein the said spike glycoprotein of SARS-CoV-2 which is expressed in adenovirus Ad5 or ChAd36 is
full-length (S) SARS-CoV-2 spike protein (SARS-CoV2-S) having nucleotide sequence of SEQ. ID No. 1,
or
subunit (S1) of SARS-CoV-2 spike protein (SARS-CoV2-S1) having nucleotide sequence of SEQ. ID No. 2,
or
full-length (S) SARS-CoV-2 spike protein (SARS-CoV2-S) having nucleotide sequence of SEQ. ID No. 3.

9. The vaccine composition as claimed in claim 8, wherein the expression of spike glycoprotein of SARS-CoV-2 in adenovirus Ad5 or ChAd36 produces below adenovirus vector constructs 1-3 (vaccine candidates 1-3):
Construct-1:
Ad5 expresses full-length(S) SARS-CoV-2 spike protein (SARS-CoV2-S)
Construct-2:
Ad5 expresses subunit (S1) of SARS-CoV-2 spike protein (SARS-CoV2-S1)
Construct-3:
ChAd36 expresses full-length(S) SARS-CoV-2 spike protein (SARS-CoV2-S).

10. The vaccine composition as claimed in claim 9, wherein said adenoviruses-based vector construct-1, construct-2, and construct-3, produce adenovirus vectors rAd5-S, rAd5-S1, and ChAd36 respectively, which are used as vaccine candidates (antigens) for preparation of vaccine for mammals for Covid-19.

11. The vaccine composition as claimed in claims 2-9, wherein said spike glycoprotein of the SARS-CoV-2 are expressed under transcriptional control.

12. The vaccine composition as claimed in claim 11, wherein said transcriptional control is any transcriptional control element introduced into the viral vector expressing spike glycoprotein of SARS-CoV-2.

13. The vaccine composition as claimed in claim 12, wherein said transcriptional control elements include but are not limited to lac operator, tet operator and other similar operators.

14. The vaccine composition as claimed in claims 11, 12 and 13, wherein said transcriptional control reduce the expression of spike glycoprotein of the SARS-CoV-2 during generation and propagation of virus vectors expressing spike glycoprotein of the SARS-CoV-2 in mammalian cells specifically developed for controlled expression of SARS-CoV-2 virus proteins under the control elements as claimed in claims 11, 12 and 13.

15. The vaccine composition as claimed in claim 14, wherein the said cells are mammalian cells developed for expression of a transcription control protein suitable to control transcription of spike glycoprotein of the SARS-CoV-2 under transcriptional elements.

16. The vaccine composition as claimed in claim 1, wherein the said immunogenic composition comprises one or more antigen(s) selected from adenovirus vectors rAd5-S, rAd5-S1, and ChAd36 in a concentration in the range between 10\hat{ }9 to 10\hat{ }12 virus particles.

17. The vaccine composition as claimed in claim 16, wherein the composition may comprise and/or formulated with or without one or more pharmaceutically acceptable excipient(s) which may be selected from buffer, cryo-protectant, salt/isotonic agent, stabilizer, diluent or carrier, propellant (for spray formulation) etc.

18. The vaccine composition as claimed in claims 16-17, wherein the composition is typically formulated as a liquid, solution, microemulsion, liposome, spray or other formulation type suitable for administration to a human subject, preferably the composition is formulated in a suitable dosage form for intranasal (I/N) administration.

19. The vaccine composition as claimed in claim 18, wherein the composition is formulated as a liquid formulation or a spray formulation, suitable for intranasal (I/N) administration.

20. The vaccine composition as claimed in claims 1-19, wherein said immunogenic composition does not induce antibodies to non-structural proteins of SARS-CoV-2, so that tests designed to differentiate vaccinated and infected humans based on the detection of antibodies to certain non-structural proteins can be employed in combination with immunization with the claimed composition.

21. The vaccine composition as claimed in claims 1-20, wherein the use of adenovirus vector concentration is between 10\hat{ }9 to 10\hat{ }12 virus particles.

22. The vaccine composition as claimed in claims 1-21, wherein the immunogenic composition comprises following ingredients:
Antigen: active ingredient adenovirus vector selected from rAd5-S or rAd5-S1 or ChAd36 or any combination thereof as antigen(s) in a concentration range between 10\hat{ }9 to 10\hat{ }12 virus particles;
Buffer: buffered with Tris-HCl or phosphate buffer or combination thereof at pH that ranges from 7.0 to 7.6 in a concentration range between 10 to 20 mM,
Cryo-protectant: Glycerol in a concentration range between 1.5 to 4.5%,
Salt: Sodium chloride for osmolarity in a concentration range between 20 to 30 mM,
Stabilizer: Magnesium chloride in a concentration range between 1 to 4 mM, and
Stabilizer (optional): Polysorbate-80 in a concentration range between 0.01 to 0.2%.

23. The vaccine composition as claimed in claim 22, wherein the immunogenic composition comprises following ingredients:
Antigen: active ingredient adenovirus vector selected from rAd5-S or rAd5-S1 or ChAd36 or any combination thereof as antigen(s) in a concentration range between 10\hat{ }9 to 10\hat{ }12 virus particles;
Tris-HCl 20 mM at pH that ranges from 7.0 to 7.6,
Glycerol 2.5%,
Sodium chloride 25 mM,
Magnesium chloride 2 mM, and
Polysorbate-80 at 0.1%.

24. A method of production of adenovirus vectors rAd5-S, rAd5-S1, and ChAd-S as claimed in claim 16, wherein the method involves lysis of the infected cells, combination of the TFF, Hollow fiber or size-exclusion chromatography.

25. A method to estimate the infectious unit by detection of the transgene (spike) expression by the adenovirus vector by chromogenic substrate but not limited to 3-Amino-9-Ethylcarbazole (AEC) or 3,3′-Diaminobenzidine (DAB) that correlates to the expression of the transgene (spike protein) which can be extrapolated to the detection of the quantity of spike expressing units in the sample.

26. A Stable immunogenic composition, wherein the one or more active ingredient adenovirus vector(s) between 10\hat{ }9 to 10′42 virus particles as antigen(s) are buffered with Tris-HCl at pH that ranges from 7.0 to 7.6 in a concentration range between 10 to 20 mM, Glycerol as cryo-protectant in a concentration range between 1.5 to 4.5%, Sodium chloride for osmolarity in a concentration range between 20 to 30 mM, Magnesium chloride as stabilizer in a concentration range between 1 to 4 mM, and with or without Polysorbate-80 as stabilizer in a concentration range between 0.01 to 0.2%.

27. The stable immunogenic composition as claimed in claim 26, wherein the composition is administered intranasally (I/N).

28. Adenovirus vector viz. rAd5-S or rAd5-S1 or ChAd36 to be used as antigen in the preparation of adenovirus viral vector-based vaccine for Covid-19 and prophylaxis and eliciting an immune response against SARS-CoV-2 and its variants in mammals, wherein the said adenovirus is adenovirus type-5 (Ad5) or Chimpanzee adenovirus 36 (ChAd36) which is engineered to express SARS-CoV-2 spike glycoprotein and its variants in mammals, and wherein the adenovirus vector comprises a vector construct as follows:
Vector Construct-1:
Ad5 expresses full-length (S) SARS-CoV-2 spike protein (SARS-CoV2-S),
Vector Construct-2:
Ad5 expresses subunit (51) of SARS-CoV-2 spike protein (SARS-CoV2-S1),
Vector Construct-3:
ChAd36 expresses full-length (S) SARS-CoV-2 spike protein (SARS-CoV2-S).

29. The adenovirus vector as claimed in claim 28, wherein the said spike glycoprotein of SARS-CoV-2 or parts/fragments thereof which is expressed in adenovirus is full length spike glycoprotein (S) or 51 subunit glycoprotein which comprises:
full-length (S) SARS-CoV-2 spike protein (SARS-CoV2-S) having nucleotide sequence of SEQ. ID No. 1,
or
subunit (S1) of SARS-CoV-2 spike protein (SARS-CoV2-S1) having nucleotide sequence of SEQ. ID No. 2,
or
full-length (S) SARS-CoV-2 spike protein (SARS-CoV2-S) having nucleotide sequence of SEQ. ID No. 3.

30. The viral vector as claimed in claims 1-3 and 28, wherein the viral vector is adenovirus which may be selected from other species, including but not limited to bovine, ovine, caprine, porcine, rhesus, chimpanzee or avian adenoviruses, or any other viral vector, in combination with or without the controlled system of expression.

31. A method of treatment and/or prophylaxis for COVID-19 and/or eliciting an immune response against SARS-CoV-2 and its variants in mammals including human subjects, wherein the said method comprises nasal administration of a vaccine formulation comprising:
a vaccine composition as claimed in claims 1-23,
or
a stable immunogenic composition as claimed in claims 26-27,
optionally, may be administered in combination with other suitable immunogenic composition which may be selected from immunogenic compositions for various types of live or replication-defective Influenza vaccine compositions.

32. The vaccine composition, immunogenic composition and method of treatment as claimed in claims 1-23, 26-27 and 31 respectively, wherein the composition or vaccine is formulated in a suitable dosage form, stored in a suitable container suitable for nasal or intranasal (I/N) application to human subjects in a suitable dose sufficient to elicit an immune response against SARS-CoV-2 and its variants in mammals.

33. The suitable dosage form as claimed in claim 32, wherein the dosage form comprises liquid of drop(s) or spray form or other similar forms suitable for intranasal (I/N) administration.

34. The suitable dose as claimed in claim 32 comprises of adenovirus between 10\hat{ }9 to 10\hat{ }12 virus particles.
